SEQUENCE LISTING (38 sequences)

SEQ ID NO: 1 - DNA, 1161 bp, Clostridium taeniosporum
atgagaaatcaatatttatataatagaaataatacaggatataatgactgtaataaaggatataatgactgtaatatttgttgcagttctaatggtgttactagagtatgtcctccaggtcctgtaggacctaagggatcaacaggagcacaaggcccaacaggggcaacaggagatagtccagttataggacctaatggaaattggtttattggtgaagtagacactggagttccagcacaaggcccaacaggagcaacaggtccatgttgtacaggcccaacaggagcgacaggagatagtccagttataggacctaatggaaattggtttattggtggagtagacactggagttccagcacaaggcccaacaggcccaacaggagcaacaggagatagtccagttataggacctaatggaaattggtttattggtgaagtagacactggagttccagcacaaggcccaacaggagcaacaggtccatgttgtacaggcccaacaggagcgacaggagatagtccagttataggacctaatggaaattggtttattggtggagtagacactggagttccagcacaaggtccaacaggcccaacaggagcaacaggagaaataggtccaacaggagcaacaggtccatgctgtacaggtccaacaggagcaacaggagaaataggtccaacaggagcaactggagttacaggccctactgcacctacaggcccatttttatctagttttattgaggtttcacgtaatacagatcaaactttgaatgctaatgatacagttgattttacacctgtaagcactgcaggaactgctcttagctttaatggtacagatactataactattaatgaaccaggtctttattatgcagatttttctgtcggtgtcactgagccagatgggccaagttcttttgctttatttgtagatggtaatataactgttgcagctccagagcctgtactaggacctaatggtggaggtaatatagcaggtggaacattaattaatgtaacaacaactccagttactattactttagttaatactagtggtgtaactagaactatctcaggagttggtggcggttctgcagctgatttacgtgtatttaaatttgcagatggaccatcagtatag

SEQ ID NO: 2 - protein, 386 aa, Clostridium taeniosporum
MRNQYLYNRNNTGYNDCNKGYNDCNICCSSNGVTRVCPPGPVGPKGSTGAQGPTGATGDSPVIGPNGNWFIGEVDTGVPAQGPTGATGPCCTGPTGATGDSPVIGPNGNWFIGGVDTGVPAQGPTGPTGATGDSPVIGPNGNWFIGEVDTGVPAQGPTGATGPCCTGPTGATGDSPVIGPNGNWFIGGVDTGVPAQGPTGPTGATGEIGPTGATGPCCTGPTGATGEIGPTGATGVTGPTAPTGPFLSSFIEVSRNTDQTLNANDTVDFTPVSTAGTALSFNGTDTITINEPGLYYADFSVGVTEPDGPSSFALFVDGNITVAAPEPVLGPNGGGNIAGGTLINVTTTPVTITLVNTSGVTRTISGVGGGSAADLRVFKFADGPSV

SEQ ID NO: 3 - DNA, 813 bp, Clostridium taeniosporum
gtggtagaattaaaagttttaaaaagtgctgatagaagctacgttttttttggaatatcaaatagaataaaatattctatctctataactaatataggggatactaaagctacctctgtaaaagtaaaagatttaatgagcaaaggagcacaatttataccaggtacttttacaataaatggatgctgccaagatataaatggaattaacagaaacattaatgtaggctcaataaaaccaggatccaatacaataataacttttgatgttgagattgtagaatataaccctccatcagaaatagtaaataaggctattgtaacttattgtgatgagaatgggaatgtattagtagctgaaagtcaagagttagtaataccaataattaaaataaatgttggaatgaaaaagacagttgataaatgtactgcaaaagttggagaaatagtaagttattctgtattaattactaataacagtaatatcaaaattgataatgttgtattttatgattcattaccaaaagaagtacaattattaccagcaagtgttttaataaatttagagcctcaatataatgaaaactttgatggtggaatacctttaggaacattaaatgcttattcaagtattatgataagtttccaagttgtaatagtttcattaccaaattcaaaattattaaagaatagttcaacaatagaatttagttataccattttagataatggaattccagtaacttctttaggtgaagcttgtagttgtgaggtaataactaaagttcttgatagtattttacaatgctaatga

SEQ ID NO: 4 - protein, 269 aa, Clostridium taeniosporum
MVELKVLKSADRSYVFFGISNRIKYSISITNIGDTKATSVKVKDLMSKGAQFIPGTFTINGCCQDINGINRNINVGSIKPGSNTIITFDVEIVEYNPPSEIVNKAIVTYCDENGNVLVAESQELVIPIIKINVGMKKTVDKCTAKVGEIVSYSVLITNNSNIKIDNVVFYDSLPKEVQLLPASVLINLEPQYNENFDGGIPLGTLNAYSSIMISFQVVIVSLPNSKLLKNSSTIEFSYTILDNGIPVTSLGEACSCEVITKVLDSILQC

SEQ ID NO: 5 - DNA, 813 bp, Clostridium taeniosporum
gtggtagatttaaaagtagtaaaaagcgctgataaaagctatatattttttggtatatcaaatagggtaaaatattctatagttataactaatgtaggagatacaaaagctaattgtgtaaaaataagagatataatgagcaaaggagcatactttataccaggcacttttacaataaatggttgctgtacagatataaatgaaattaatagcaatattaatataggatcaataaaaccaggttcaaatataatagttacttttgatgttgaggtagtaaagtataatccaccatcacaaatagaagatcaagctatagtgacttattgtgacgaaaatactaatattttaactgcttacagtcaagtgttaacaatacctgtaattagaataaatgttgggatgaaaaagacagttgataaatgcactgcaaaagttggtgaaatagtaagttattctgtattaattactaataatagtaatatcaaaattgacgatgttgtattttatgattcattaccagaagaagtacaattattaccagcaagtgttttaataaatttagagcctcaatataatgaaaactttgatggtggaatacctttaggaacattaaatgcttattcaagtattatgataagtttccaagttgtaatagtttcattaccgaattcgaaattattaaagaatagttcaacaatagaatttagttataccattttagataatggaattccagtaacttctttaggcgaagcttgtagttgtgaggtaataactaaagttcttgatagtattttacaatgctaatga

SEQ ID NO: 6 - protein, 269 aa, Clostridium taeniosporum
MVDLKVVKSADKSYIFFGISNRVKYSIVITNVGDTKANCVKIRDIMSKGAYFIPGTFTINGCCTDINEINSNINIGSIKPGSNIIVTFDVEVVKYNPPSQIEDQAIVTYCDENTNILTAYSQVLTIPVIRINVGMKKTVDKCTAKVGEIVSYSVLITNNSNIKIDDVVFYDSLPEEVQLLPASVLINLEPQYNENFDGGIPLGTLNAYSSIMISFQVVIVSLPNSKLLKNSSTIEFSYTILDNGIPVTSLGEACSCEVITKVLDSILQC

SEQ ID NO: 7 - protein, 6 aa, Artificial Sequence
  Synthetic peptide
KFYTNK

SEQ ID NO: 8 - DNA, 28 bp, Artificial Sequence
  Synthetic primer
aacaaaatatattatgtaatcatataat

SEQ ID NO: 9 - DNA, 29 bp, Artificial Sequence
  Synthetic primer
ttgacaatataatttagtattattaaaat

SEQ ID NO: 10 - DNA, 21 bp, Artificial Sequence
  Synthetic primer
atggtwgaattaaaagtwtta

SEQ ID NO: 11 - DNA, 23 bp, Artificial Sequence
  Synthetic primer
atcataaaanacnacattatcta

SEQ ID NO: 12 - DNA, 17 bp, Artificial Sequence
  Synthetic primer
atgagnaatcaatattt

SEQ ID NO: 13 - DNA, 26 bp, Artificial Sequence
  Synthetic primer
gtagcagcatcwswattaccaactcc

SEQ ID NO: 14 - protein, 6 aa, Artificial Sequence
  Synthetic peptide
GYNDCN

SEQ ID NO: 15 - DNA, 4101 bp, Clostridium taeniosporum
tacttttatttatagatgtaaagttctgttagaaaggagaggaagttatggctgttaataatatagatgaatgtaatagtaatataatattgcaaaaaagtgtagataaagaaaatgcagtagtaggagatattataaaatataatatatctattattaatcaaggtgatatattagttaataatgttacaattatagatactttagtaagtgaattagaatttaaaatgggtagtgtagttttatgtgatataccattaccagatgagagtgtattatctggaattgatattggatgtcttaaaccaggacaaattaagacattaacatttaaagctagagttatatgcaggcctagtagcggatatatagaaaatagtgctattgcaaagtttaattatgatgccaatttagataattgtttgaagacaatggatgagactagtaatatagtatctataaaaatagatgttgctgaagtaaaaataataaaaactgcagataaaaaaatagcatctagaggagatattgtatcatatgaagttaaacttataaatgttggaactttggaagctagaaatattttatttaccgataagttaccgcctgaagtttgtttagttgatgatacttttgaagtgaatggaaacccaattaataatataggtcatgatatagatgtatacgtaggctctataaatcctggagaatatattatagtaacttatagagttattgttaaatcttcaaattgcagtggattaattataaacaaagctttcagtaaattttgattataatatatgcaatggggattttggtaaaaaaatttctagatgtaatgaatatgaaacaagtgaagttagtcttggaataagtacgtttaaacaaatatcaatagatgataaattatgtataccatgtgtaaaaccagatatagaagaaataaatgatatgaaagtggaagccaaaatattagaatgtcatgttataagcactccatcaattatatcaaatgaaggacaaatattatcaggatataaattaattgttagaggtgtgttagaggaattagttgaatatacatctgctactgaagaacaatcagtacattcagcacattattcaataccattttcaagttttttaatattaccatcaacttatgtagttggaagtaaaatagatatagagggtaaagttgaagatatttattataagaaaatagattctagatgtttctttaaaaatattacaattttaattaatgctaaaataatgtcttgttaatcaataaaatatttgtaacaaaatatattatgtaatcatataatatattatctataaagcaaatcaaaacaataattttttggttattagtattggttataaatgctttgaaaggagaaaaatagcgcgtggtagaattaaaagttttaaaaagtgctgatagaagctacgttttttttggaatatcaaatagaataaaatattctatctctataactaatataggggatactaaagctacctctgtaaaagtaaaagatttaatgagcaaaggagcacaatttataccaggtacttttacaataaatggatgctgccaagatataaatggaattaacagaaacattaatgtaggctcaataaaaccaggatccaatacaataataacttttgatgttgagattgtagaatataaccctccatcagaaatagtaaataaggctattgtaacttattgtgatgagaatgggaatgtattagtagctgaaagtcaagagttagtaataccaataattaaaataaatgttggaatgaaaaagacagttgataaatgtactgcaaaagttggagaaatagtaagttattctgtattaattactaataacagtaatatcaaaattgataatgttgtattttatgattcattaccaaaagaagtacaattattaccagcaagtgttttaataaatttagagcctcaatataatgaaaactttgatggtggaatacctttaggaacattaaatgcttattcaagtattatgataagtttccaagttgtaatagtttcattaccaaattcaaaattattaaagaatagttcaacaatagaatttagttataccattttagataatggaattccagtaacttctttaggtgaagcttgtagttgtgaggtaataactaaagttcttgatagtattttacaatgctaatgaaaaacaaagtgatgattttaattaagaggagtgttattatatgagaaatcaatatttatataatagaaataatacaggatataatgactgtaataaaggatataatgactgtaatatttgttgcagttctaatggtgttactagagtatgtcctccaggtcctataggtcctaagggatcaacaggcccaacaggagcaacaggagaaataggtccaacaggtccaattgggttaacaggagcaacaggtccatgttgtacaggtccaacaggagcaacaggagtaaccggagcaataggagcaacaggtccaattgggttaacaggggcaacaggtccatgttgcacaggtccaacaggagcaacaggagtaaccggagcaataggagcaacaggtccaattgggttaacaggagcaacaggtccatgttgtacaggcccaacaggagcaacaggagtaacaggacaaacaggagaaacaggagtaaccggagcaaccggagaaacaggagcaaccggagcaaccggagcaaccggtccattaataacttcaaataatgcatttatagtaagtgaacagccaactcaagcaatagcagatacaactcctattccacttactacaaatgtagtaattaatggaacagcaattactcatactgcaggatcaactgatattgtattagcaccaaatcaacaatatttggtcgtttatagagccgttgggtttgatacaggtttaccagcaggtagttattataatcttaatttaacattagatggaattttggttggtggaacagatgcaggttctacttcaactggaacacctcctagtccagaaatgactggaacagctattgtaaacactggagctggaccaaatattttacaactaataaatgcatctggaggaactagaacatttgatgacgttcaaatttctattgtaaaattagcttaataaattagatatagattaaatagagttgtatttgatttataaaaccaatgaatggggctgtcgcactaaaaaatagtgcgaagctcttttttagtgcaaaaaaataaatctcaaactcctcagagttatatcaattaaaacttcaattaaatattaattatatgataccagttaatgattcagtacgtttactaagtgaatttgtagaggagatggatttagcagatttgtattcgacttattcctgtattaaaaggtttatataatttagaaaatccaagactctatgggcaagccccataagaacgtcatacaacaataacatctttaaatacagtatctactagaggacaatcaggattaatacttctagttagtgtgggagatagagtgctatttgtaagttgacaaagaatgacagcaacataaaagaaaaaatttatggtacatattttgattaagtttcatattataaataagtgatttttttaataggaatattgttttcttgttaaatttattctattttataagattgataagattaatgttcatataaattaacagaacgattttaaatgaagggagttattatatgggaaagaaaataacactaagtttatgcatgatagtaaaagatgaagaaaaaacattagagcgttgtttaaattcagtaaaatcttttattgatgaaattataattgttgatactggatctaaggattctacaaaagatattgcaaaaaaatttaatgctaaaatttatgatttcaaatgggtaaatgatttttctaaagctagaaatttttcatttagtaaagcaactagtgactatattttatggttagatggtgatgattttatagatcaaaagaatataaaaattattgagaatttattatcaag

SEQ ID NO: 16 - DNA, 2642 bp, Clostridium taeniosporum
aattactaataacagtaatatcaaaattgataatgttgtattttatgattcattaccaaaagaagtacaattattaccagcaagtgttttaataaatttagagcctcaatataatgaaaactttgatggtggaatacctttaggaacattaaatgcttattcaagtattatgataagtttccaagttgtaatagtttcattaccaaattcaaaattattaaagaatagttcaacaatagaatttagttataccattttagataatggaattccagtaacttctttaggtgaagcttgtagttgtgaggtaataactaaagttcttgatagtattttacaatgctaatgaaaaacaaagtgatgattttaattaagaggagtgttattatatgagaaatcaatatttatataatagaaataatacaggatataatgactgtaataaaggatataatgactgtaatatttgttgcagttctaatggtgttactagagtatgtcctccaggtcctgtaggacctaagggatcaacaggagcacaaggcccaacaggggcaacaggagatagtccagttataggacctaatggaaattggtttattggtgaagtagacactggagttccagcacaaggcccaacaggagcaacaggtccatgttgtacaggcccaacaggagcgacaggagatagtccagttataggacctaatggaaattggtttattggtggagtagacactggagttccagcacaaggcccaacaggcccaacaggagcaacaggagatagtccagttataggacctaatggaaattggtttattggtgaagtagacactggagttccagcacaaggcccaacaggagcaacaggtccatgttgtacaggcccaacaggagcgacaggagatagtccagttataggacctaatggaaattggtttattggtggagtagacactggagttccagcacaaggtccaacaggcccaacaggagcaacaggagaaataggtccaacaggagcaacaggtccatgctgtacaggtccaacaggagcaacaggagaaataggtccaacaggagcaactggagttacaggccctactgcacctacaggcccatttttatctagttttattgaggtttcacgtaatacagatcaaactttgaatgctaatgatacagttgattttacacctgtaagcactgcaggaactgctcttagctttaatggtacagatactataactattaatgaaccaggtctttattatgcagatttttctgtcggtgtcactgagccagatgggccaagttcttttgctttatttgtagatggtaatataactgttgcagctccagagcctgtactaggacctaatggtggaggtaatatagcaggtggaacattaattaatgtaacaacaactccagttactattactttagttaatactagtggtgtaactagaactatctcaggagttggtggcggttctgcagctgatttacgtgtatttaaatttgcagatggaccatcagtatagaaaatatataaattgaaaatataatttagtattattaaaatatagataatcaaaaataaattttaaggcacatgttttatttaagacatgtgctttttagtaatataaattattagtaaataaggtatcagtgttatagaaatgaagaagaacaaatggatataagaaatcatataatgatttatgatgcttagaataaataaactaaatttagtttatttataattagaaatgctttagaaaggagaaaaataacacgtggtagatttaaaagtagtaaaaagcgctgataaaagctatatattttttggtatatcaaatagggtaaaatattctatagttataactaatgtaggagatacaaaagctaattgtgtaaaaataagagatataatgagcaaaggagcatactttataccaggcacttttacaataaatggttgctgtacagatataaatgaaattaatagcaatattaatataggatcaataaaaccaggttcaaatataatagttacttttgatgttgaggtagtaaagtataatccaccatcacaaatagaagatcaagctatagtgacttattgtgacgaaaatactaatattttaactgcttacagtcaagtgttaacaatacctgtaattagaataaatgttgggatgaaaaagacagttgataaatgcactgcaaaagttggtgaaatagtaagttattctgtattaattactaataatagtaatatcaaaattgacgatgttgtattttatgattcattaccagaagaagtacaattattaccagcaagtgttttaataaatttagagcctcaatataatgaaaactttgatggtggaatacctttaggaacattaaatgcttattcaagtattatgataagtttccaagttgtaatagtttcattaccgaattcgaaattattaaagaatagttcaacaatagaatttagttataccattttagataatggaattccagtaacttctttaggcgaagcttgtagttgtgaggtaataactaaagttcttgatagtattttacaatgctaatgaaaaacaaagtgatgattttaa

SEQ ID NO: 17 - protein, 6 aa, Artificial Sequence
  Synthetic peptide
GPTGAT

SEQ ID NO: 18 - protein, 5 aa, Artificial Sequence
  Synthetic peptide
GVPAQ

SEQ ID NO: 19 - protein, 5 aa, Artificial Sequence
  Synthetic peptide
GPCCT

SEQ ID NO: 20 - protein, 20 aa, Artificial Sequence
  Synthetic peptide
GDSPVIGPNGNWFIGEGVDT

SEQ ID NO: 21 - protein, 307 aa, Clostridium taeniosporum
MRNQYLYNRNNTGYNDCNKGYNDCNICCSSNGVTRVCPPGPIGPKGSTGPTGATGEIGPTGPIGLTGATGPCCTGPTGATGVTGAIGATGPIGLTGATGPCCTGPTGATGVTGAIGATGPIGLTGATGPCCTGPTGATGVTGQTGETGVTGATGETGATGATGATGPLITSNNAFIVSEQPTQAIADTTPIPLTTNVVINGTAITHTAGSTDIVLAPNQQYLVVYRAVGFDTGLPAGSYYNLNLTLDGILVGGTDAGSTSTGTPPSPEMTGTAIVNTGAGPNILQLINASGGTRTFDDVQISIVKLA

SEQ ID NO: 22 - protein, 18 aa, Artificial Sequence
  Synthetic peptide
GVTGAIGATGPIGLTGAT

SEQ ID NO: 23 - DNA, 23 bp, Artificial Sequence
  Synthetic primer
gagagtttgatcctggctcagga

SEQ ID NO: 24 - DNA, 20 bp, Artificial Sequence
  Synthetic primer
aaggaggtgatccagccgca

SEQ ID NO: 25 - DNA, 1464 bp, Clostridium taeniosporum
cgaacgctggcggcgtgcctaacacatgcaagtcgagcgatgaagctccttcgggagtggattagcggcggacgggtgagtaacacgtgggcaacctgcctcatagagagggatagccttccgaaaggaagattaatacctcataagattgtagtttcgcatgaaacagcaataaaaggagcaatccgctatgagatgggcccgcgtcgcattagctagttggtaaggtaatggcttaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaaactctgtctttggggacgataatgacggtacccaaggaggaagccacggctaactacgtgccagcagccgcggtaatacgtaggtggcaagcgttgtccggatttactgggcgtaaagggagcgtaggtggatatttaagtgggatgtgaaatactcgggcttaacctgggtgctgcattccaaactggatatctagagtgcaggagaggaaagtggaattcctagtgtagcggtgaaatgcgtagatattaggaagaacaccagtggcgaaggcgactttctggactgtaactgacactgaggctcgaaagcgtggggagcgaacaggattagataccctggtagtccacgccgtaaacgatgaatactaggtgtaggagctgtcatggcttctgtgccgccgctaacgcattaagtattccgcctggggagtacggtcgcaagattaaaactcaaaggaattgacgggggcccgcacaagcagcggagcatgtggtttaattcgaagcaacgcgaagaaccttacctagacttgacatctcctgaattactcttaatcgaggaagtcgcttcggcgacaggaagacaggtggtgcatggttgtcgtcagctcgtgtcgtgagatgttgggttaagtcccgcaacgagcgcaacccttattgttagttgctaccatttagttgagcactctagcgagactgccgtggttaacgcggaggaaggtggggatgacgtcaaatcatcatgccccttatgtctagggctacacacgtgctacaatggctggtacagagagacgcaataccgtgaggtggagctaaactttaaaaccagtctcagttcggattgtaggctgaaactcgcctacatgaagctggagttgctagtaatcgcgaatcagaatgtcgcggtgaatacgttcccgggccttgtacacaccgcccgtcacaccatgagagttggcaatacccaaagttcgtgagctaacccgtaagggaggcagcgacctaaggtagggtcagcgattggggtgaagtcgtaacaaggtagccgtaggagaacc

SEQ ID NO: 26 - protein, 21 aa, Artificial Sequence
  Synthetic peptide
MVELKVLXSADRSYVFFGIXN

SEQ ID NO: 27 - protein, 17 aa, Artificial Sequence
  Synthetic peptide
MRNQYLXNRNNTGTYND

SEQ ID NO: 28 - protein, 7 aa, Artificial Sequence
  Synthetic peptide
MKFYTNK

SEQ ID NO: 29 - protein, 12 aa, Artificial Sequence
  Synthetic peptide
LDNVVFYDSLPK

SEQ ID NO: 30 - protein, 12 aa, Artificial Sequence
  Synthetic peptide
YSLSLTNIGDTK

SEQ ID NO: 31 - protein, 8 aa, Artificial Sequence
  Synthetic peptide
VFFGLSNR

SEQ ID NO: 32 - protein, 14 aa, Artificial Sequence
  Synthetic peptide
VSYSVLLTNNSNLK

SEQ ID NO: 33 - protein, 8 aa, Artificial Sequence
  Synthetic peptide
VLTIPVIR

SEQ ID NO: 34 - protein, 8 aa, Artificial Sequence
  Synthetic peptide
PPGPVGPK

SEQ ID NO: 35 - protein, 11 aa, Artificial Sequence
  Synthetic peptide
YSLSLTTLSLP

SEQ ID NO: 36 - protein, 8 aa, Artificial Sequence
  Synthetic peptide
VFFGLESS

SEQ ID NO: 37 - protein, 9 aa, Artificial Sequence
  Synthetic peptide
SYSVNLTNG

SEQ ID NO: 38 - DNA, 28 bp, Artificial Sequence
  Synthetic primer
aacaaatggatataagaaatcatataat

(19) United States
(12) Patent Application Publication
(10) Pub. No.: US 2012/0014993 A1
(43) Pub. Date: Jan. 19, 2012

(54) CLOSTRIDIUM TAENIOSPORUM SPORES AND SPORE APPENDAGES AS SURFACE DISPLAY HOSTS, DRUG DELIVERY DEVICES, AND NANOBIOTECHNOLOGICAL STRUCTURES

(76) Inventors: James R. Walker, Austin, TX (US); Alexandra Blinkova, Austin, TX (US); Philip Tucker, Austin, TX (US)

(21) Appl. No.: 13/163,209

(22) Filed: Jun. 17, 2011

Related U.S. Application Data
(60) Provisional application No. 61/355,793, filed on Jun. 17, 2010.

(51) Int. Cl. A61K 39/08; A61P 37/00; G01N 33/554; C40B 30/04; C12N 1/20; A61K 35/74

(52) U.S. Cl. 424/247.1; 435/252.7; 424/93.41; 435/7.32; 506/9

(57) ABSTRACT

The present disclosure relates to spore surface display compositions comprising a spore having at least one nucleic acid sequence encoding for at least one polypeptide and operable to express the polypeptide on a surface of the spore. In some embodiments, the displayed polypeptide is displayed with a spore carrier protein. In some embodiments, the spore may be derived from a Clostriduim sp. such as Clostriduim taeniosporum. Spore display compositions of the disclosure may include vaccines, fusion proteins, drug delivery devices, systems for generating an antibody to an antigen/peptide expressed on a spore surface, an anticancer drug, an immobilized enzyme system, a system for serological reagent preparation, a contaminant removal system, a biocatalysis system, a screening platform, a nanotechnology platform, a bioanalytical sensor, a molecular electronic system and/or a signal processing system. Methods for making and using these compositions are described.

This application claims priority to U.S. Application No. 61/355,793 filed on Jun. 17, 2010, the entire disclosure of which are specifically incorporated herein by reference in its entirety without disclaimer.

### BACKGROUND

1. Field of the Invention

The present invention relates generally to the field of protein engineering. More particularly, it concerns a surface display system for expressing peptides and proteins on the surface of a microorganism, especially of a spore.

2. Description of Related Art

Several surface display systems have been used for expressing peptides and small proteins on the surface of phage and/or bacteria. In bacterial surface display systems, target proteins or peptides are typically fused to bacterial cell surface proteins such as outer membrane proteins of Gram negative bacteria, S-layer proteins of Gram positive bacteria, pilin, flagelllin or sp flagellin, to obtain fusion proteins that may be expressed on the surface of a bacterium. In these systems, target proteins are synthesized in the cytoplasm and must traverse the cytoplasmic membrane, the periplasmic gel, and the outer membrane (of Gram negative hosts) prior to being displayed. These steps limit the size, enzyme activity and/or folding of the target protein.

Various phage surface display systems have been developed, but are limited by the maximum size and copy number of an expressed target protein. For example, in some filamentous phage systems, peptides of only about 6-10 amino acid residues may be displayed with phage major coat proteins. Other phage display systems display full-length polypeptides on minor phage coat proteins, however, the proteins are displayed at a very low copy number. Bacterial display systems are also limited by the need for a fusion protein to pass through the secretion system of the host which may pose problems of toxicity for the host and may also affect the correct folding of the displayed protein.

### SUMMARY

The present disclosure relates to compositions comprising spores (e.g., of Clostridium taeniosporum) configured to have one or more nucleic acids operable to synthesize one or more proteins that in some embodiments may be expressed on the surface of a spore. In some embodiments, compositions of the disclosure may include but are not limited to spore surface display systems, fusion proteins, drug delivery devices, vaccines, anticancer drugs, screening platforms, nanotechnology platforms and tools. The disclosure, in some embodiments, also describes methods for making compositions of the disclosure, methods for screening, methods for generating and/or isolating proteins, methods for delivering drugs and therapeutic methods.

As protein surface display systems have many applications, a display system with the ability to produce a variety of functional proteins having a high copy number is desired. The present disclosure, in some embodiments relates to a spore surface display system that provides several advantages.

In one embodiment, the present disclosure describes a spore surface display system comprising a spore configured (e.g., engineered) to have at least one nucleic acid encoding a polypeptide of interest, the spore also being operable to express (e.g., transcribe, translate, translocate and/or assemble) the polypeptide of interest on the surface of the spore. In some embodiments, a spore may be from a Clostridium sp. In some embodiments the Clostridium sp. may be a non-toxic species of Clostridium.

In some embodiments, a spore display system of the disclosure may express a polypeptide of interest as a fusion protein. A fusion protein and/or a surface displayed system of the disclosure may comprise a protein of interest fused with a spore protein or spore peptide. Spore proteins are described in detail in sections below and may include an exosporial protein, a spore coat protein, and/or a spore appendage protein.

Spore display systems of the disclosure may be used to display any polypeptide of interest including, but not limited to, small polypeptides, larger proteins, epitopes, antigens, antibodies, hormones, enzymes, anticancer proteins, therapeutic proteins or peptides, and/or peptides or proteins that have an affinity for another biomolecule. Accordingly a nucleic acid encoding any polypeptide of interest may be introduced into a spore according to the present teachings to obtain a spore display system.

The disclosure also describes compositions such as fusion proteins, drug delivery devices, vaccines, anticancer drugs, screening platforms, nanotechnology platforms and tools.

For example, a spore surface display composition may be used as a vaccine that may comprise at least one immunogenic epitope derived from an infectious agent or a disease causing agent to be expressed and displayed on the surface of a spore. In another example, a spore composition of the discourse may comprise an anticancer protein displayed on the surface of a spore optionally having a tumor marker co-expressed. In one example, a composition of the disclosure may be used to bind or associate with a drug. In some examples, proteins or peptides may be displayed in regular arrays to form nanotechnology tools. In some examples, proteins or peptides may be displayed to form a screening or analytical tool. The disclosure is not limited to these examples and many other compositions are possible some of which are described in sections ahead.

In some embodiments, a composition of the disclosure may further be formulated as a pharmaceutical formulation that may be suitable for administration to a subject. In some embodiments, pharmaceutical compositions of the disclosure may also comprise adjuvants, other immunoactive agents, other therapeutic agents, or pharmaceutical carriers and/or solvents. Pharmaceutical formulations are described later in this specification.

Some embodiments of the disclosure relate to a spore surface display system, which involves the introduction of a plasmid into a spore. The plasmid is engineered to preferably have a spore promoter or mother cell late promoter operably linked to one or more nucleic acids encoding a polypeptide and at least one carrier, so that the spore promoter is operable to generate a fusion protein comprising the polypeptide and the carrier protein to be expressed on the surface of the spore. In some embodiments, the carrier protein is a spore coat protein, an exosporial protein, or a spore appendage protein. Of course, the use of any promoter operable in Clostridium is contemplated in broader aspects of the invention.

In some embodiments, the plasmid used in the spore surface display system may further comprise a general transcription promoter which directs the expression of the fusion protein in a suitable host, such as E. coli. The “general transcription promoter” refers to a transcription promoter conventional in the art. The plasmid comprising both a spore promoter or mother cell late promoter and a general transcription promoter may be engineered to introduce mutations into the carrier gene in E. coli, or other suitable hosts, then shuttled into Clostriduim taeniosporum and the fusion proteins are expressed on the surface of the spore. In some examples, the mutations may comprise insertion of a polypeptide gene into the carrier gene.

The disclosure also describes the use of the disclosed spore surface display system in combination with the method of Anchored Periplasmic Expression (APEx).

In some embodiments, the spore surface display system described above may have immunologic applications, such as forming a composition for a vaccine.

Some embodiments of the disclosure relate to methods of making spore surface display compositions and/or other compositions as set forth above. The disclosure, in some embodiments, also describes methods for making fusion proteins. In some embodiments, the present disclosure describes methods for generating proteins. As spore based compositions of the disclosure produce high copies of proteins, systems of the disclosure may be used to generate large quantities of protein. In some embodiments, such a method may further comprise isolating one or more expressed protein. As proteins are expressed on the surface of spores, which in some embodiments may have very few indigenous spore proteins, isolation of exogenous protein may produce substantially pure isolated protein.

In some embodiments, the disclosure describes methods for screening for biomolecules (e.g., drugs, antibodies, antigens) of interest and identifying biomolecules of interest using a composition of the disclosure. The present disclosure also describes methods for making nanobiotechnology tools that may in some embodiments comprise repeats of arrays of one or more protein. In some embodiments, compositions of the disclosure may be used for analyzing protein structure.

Some embodiments of the disclosure relate to methods for delivering drugs using compositions of the disclosure. Some embodiments of the disclosure relate to therapeutic methods comprising administering to a patient or subject in need a therapeutic spore surface display formulation of the disclosure.

In some embodiments, therapeutic methods may include delivering a therapeutic protein or a therapeutic agent attached to a protein, wherein the protein is displayed on the compositions of the disclosure. In some embodiments, therapeutic methods for treating solid tumors that have a hypoxic environment are described.

A therapeutic formulation of the disclosure, such as but not limited to, a vaccine, a therapeutic protein, and/or an anticancer composition, of the disclosure may be administered to a subject via any suitable delivery method. Details regarding pharmaceutical formulations and therapeutic delivery methods are provided in the section entitled Detailed Description.

Certain embodiments of the disclosure may provide one or more technical advantages. A technical advantage of one embodiment may include proteins or polypeptides of interest having a higher copy number and/or structure and function very similar to a native polypeptide of interest. Without being limited to theory, this may be due to the synthesis and assembly into spore coat/spore appendage in the mother cell cytoplasm, thereby reducing the number of translocations and/or environmental changes and/or folding changes a polypeptide of interest may be subject to.

A technical advantage of one embodiment may include the inert nature of spores and their appendages which may reduce the effect of a fusion protein on the viability of a bacterial host. A technical advantage of one embodiment may include the large size of spore appendages which innately contain several thousand monomers of proteins which may allow many several thousand copies of a fusion protein comprising a polypeptide of interest to be synthesized or expressed on the surface.

A technical advantage of one embodiment may include immunologic applications, such as vaccines and other treatments that may rely on antigen or antibody presentation. As polypeptides of interest are displayed at high copy number, more antigen (for example) may be presented to obtain a superior immune response. A technical advantage of one embodiment may include the ability to analyze structure of a protein or polypeptide of interest that is displayed on a spore surface with multicopy spore proteins forming an array of a protein of interest. A technical advantage of one embodiment may include use of an array of a protein or polypeptide of interest that is displayed on a spore surface for a variety of screening or nanotechnology applications.

Various embodiments of the disclosure may include none, some, or all of the above technical advantages. One or more other technical advantages may be readily apparent to one skilled in the art from the figures, descriptions, and claims included herein.

### BRIEF DESCRIPTION OF THE DRAWINGS

For a more complete understanding of the present disclosure and its features and advantages, reference is now made to the following description, taken in conjunction with the accompanying drawings, in which:

FIG. 1 shows a three-dimensional protein model of a portion of a C. taeniosporum spore appendage, according to one example embodiment, which was assembled from small complexes (fibrils) consisting of three major protein components which assemble into a spherical head linked to a long, thin tail.

FIGS. 2A-2F show scanning electron microscopy (SEM) and transmission electron microscopy (TEM) analysis of C. taeniosporum spore and appendage structure, wherein FIG. 2A shows SEM of spore with ribbon-like appendages; FIG. 2B shows TEM of a negatively-stained longitudinal section of a C. taeniosporum spore from which the appendages had been sheared; FIG. 2C shows TEM of appendages; FIG. 2D shows a higher magnification of a portion of one appendage showing the surface comprised of parallel rows of beads and the edge according to one example embodiment; FIG. 2E shows a higher magnification of the appendage edge and fibrils of FIG. 2D; and FIG. 2F shows an additional TEM view of parallel rows of beads which form an appendage surface, according to one example embodiment.

FIGS. 3A-3D show an AFM study of appendage structure wherein FIG. 3A shows approximately one-half of an appendage shown in three-dimensional view; FIG. 3B shows an appendage with fibrils forming a hair-like nap around the edges in three dimensions; FIG. 3C shows three dimensional images of individual fibrils; and FIG. 3D shows individual fibrils on the mica surface in three dimensions showing spherical heads (nearly white) and long and thin tails, according to one example embodiment.

FIG. 4 shows an SDS-PAGE analysis gel of appendage proteins, before and after deglycosylation, solubilized by boiling in dithiothreitol-containing buffer, according to one example embodiment.

FIGS. 5A-5D shows organization of chromosomal regions containing genes for P29, GP85 and possibly related proteins, wherein in FIG. 5A numbers indicate nucleotide pairs; ORF positions and directions of transcription are indicated by arrows, Pσ^{A} and Pσ^{K} are putative sigma A- and K-dependent promoters, respectively and ORF 4 and P29c are incomplete; FIG. 5B shows characteristics of ORF1, P29a and P29b, CL2 and GP85 proteins deduced from the DNA sequences where numbers indicate amino acid residues, arrows indicate repeat regions and white bars indicate domains of unknown function 11 (DUF11) and the black dots in P29b indicate residues different from those in P29a and in CL2 and GP85, white bars indicate collagen-like regions (CLR), the medium dark bars indicate 39 identical N-terminal residues and the black and darker bars indicate regions with no significant homology; FIG. 5C shows putative promoter and SD regions upstream of the P29a and P29b genes and black and medium dark numbers indicate by and amino acid residues, respectively and C-termini of the ORF2 and GP85 genes are indicated and N-termini of the P29a and b genes are indicated medium dark and putative Γ^{K}- and σ^{A}-dependent promoters are indicated and consensus sequences are indicated by CONSEN and putative SD regions are shown as well in medium dark; and FIG. 5D shows N-terminal 240 and 168 residues of GP85 and CL2 proteins showing collagen-like regions are residues 40-240 and 40-168 in GP85 and CL2, respectively where the dots indicate gaps, the N-terminal 39 residues (blue) are identical and contain the sequence GYNDCN repeated (underline) and residues 40-48 are identical except for positions 42, and other repeat regions are indicated by different colors; according to one example embodiment.

FIGS. 6A-6D show the effects of injecting mice with C. taeniosporum spores or isolated appendages. FIGS. 6A and 6B show IgG or IgM responses of mice injected with C. taeniosporum spores, respectively. FIGS. 6C and 6D show IgG or IgM responses of mice injected with isolated appendages, respectively.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

It should be understood at the outset that, although example implementations of embodiments of the disclosure are illustrated below, the present disclosure may be implemented using any number of techniques, whether currently known or not. The present disclosure should in no way be limited to the example implementations, drawings, and techniques illustrated below.

In one embodiment, the present disclosure describes a spore surface display system comprising a spore configured (e.g., engineered) to have at least one nucleic acid encoding a polypeptide of interest, the spore further operable to express (e.g., transcribe, translate, translocate and/or assemble) the polypeptide of interest on the surface of the spore.

Accordingly, a nucleic acid encoding a polypeptide of interest may be introduced by recombinant DNA technology into a spore according to the present teachings. One of ordinary skill in the art with the benefit of this disclosure will be able to determine suitable methods of introducing a nucleic acid encoding a polypeptide of interest into a spore using recombinant DNA technology, including, but not limited to, cloning the nucleic acid into a plasmid known to be stable in the spore and transformation of the spore with the clone, and also gene replacement by which the nucleic acid is recombined into the spore chromosome. In some embodiments, a spore may be operable to express a polypeptide of interest onto its surface by means of a carrier protein or peptide that may be co-expressed with the protein of interest. A carrier protein or peptide may typically be a spore protein or peptide that is expressed on the surface of the spore.

A carrier protein or peptide may also be referred to herein as a spore protein or peptide and may include, but is not limited to, an exosporial protein, a spore coat protein, and/or a spore appendage protein. Some exemplary spore proteins and peptides are described in sections below. In some embodiments, smaller peptide fragments or combinations of one or more such spore proteins may be co-expressed as fusion proteins with a polypeptide of interest. In some embodiments, a spore protein or spore peptide may be derived from a spore protein and may be for example about 40% to about 99% identical to a spore protein or peptide.

A spore as used herein may include any type of bacterial spore or fungal spore. Sporulation in bacteria involves a well-regulated developmental pathway in which vegetatively growing cells sense approaching starvation and respond by forming inert, durable endospores. Vegetative cells differentiate and produce two compartments: 1) the forespore which will become the spore; and 2) the mother cell. Sporulation pathways involve regulated, compartment-specific synthesis of proteins in a logical temporal order and coordination of development within both the compartments by crosstalk between the developing forespore and the mother cell. Spore coat protein synthesis, one of the last stages of sporulation, occurs within the mother cell, but the proteins assemble into a coat around the developing forespore.

Some example embodiments of the present disclosure are described using spores of an anaerobic bacterium, Clostridium taeniosporum. However, one of skill in the art with the benefit of this disclosure may use the present teachings effectively with other species known to produce spores or appendages. For example, spores from bacteria such as Bacillus sp., including but not limited to, B. subtilis, B. megaterium, B. thuringiensis and B. cereus or other Clostridium sp., including, but not limited to, C. cochlearium, C. bifermentans, and C. sordelli. Accordingly, the present disclosure is not limited to Clostridium taeniosporum spores and may be modified to be used with other spores in light of the teachings herein.

Some embodiments may be benefited by the use of non-pathogenic bacterial strains such as for example therapeutic compositions and therapeutic or medical applications of the disclosure. The present inventors have performed phylogenetic analysis including 16S rDNA gene sequence analysis and determined that C. taeniosporum is non-pathogenic although it is closely related to C. botulinum which produces a neutotoxin. In the section entitled Examples described in later sections of this application, genomic DNA of C. taeniosporum was tested for the presence of botulinal toxin genes by Polymerase Chain Reaction (PCR) and all the tests were negative while control tests with C. botulinum DNA were positive. In addition, mouse neurotoxin bioassays using cultures and culture supernatants of C. taeniosporum were also negative. Accordingly, some embodiments of the disclosure may use spores derived compositions derived from non-pathogenic bacteria such as but not limited to C. taeniosporum, C. bifermentans, and C. sordelli.

Bacterial endospores formed by members of the Clostridium and Bacillus subphylum typically comprise a dehydrated core containing a chromosome, a double membrane layer, a peptidoglycan cortex, and a proteinaceous coat. These structures allow spore survival in extremely harsh conditions. Some Bacillus sp. and Clostridium sp. spores may be enclosed in a sac-like exosporium and may contain spore proteins such as immunodominant antigens on the spore surface (e.g., B. anthracis). Synthesized by the mother cell concomitantly with the cortex and coat, exosporia have a highly structured, paracrystalline basal layer with an external hair-like nap. B. cereus and B. anthracis exosporia have at least 11 proteins, some of which are enzymes and some of which are B. subtilis spore coat homologs. A major Bacillus exosporium component is a highly glycosylated collagen homolog (BclA) containing Gly-X-Y repeats (GXY) as in collagen where the X and Y are often proline and hydroxyproline.

Many spores also have external appendages in the form of ribbons, pili, feathers, brushes, tubules or swords. Spore appendages were discovered by Krasil'nikov et al. (1963; 1968) who isolated and described 19 new Clostridium and Bacillus species, from soil and silt of the former U.S.S.R. and Burma, all of which formed spores with appendages of various types.

Exosporia and appendages are produced separately. Some bacterial species produce one or the other and some produce both. For example, commonly studied strains of B. cereus form exosporia only while some C. botulinum type E strains form pilus-like appendages but no exosporia, and some strains of C. bifermentans produce both pilus-like appendages and exosporia. Appendage formation is also strain-dependent. Some strains of B. cereus, C. bifermentans and C. sordelli produce appendages whereas other strains of those species do not. Moreover, appendages of different types are formed by different strains of the same species. Four different appendage structures are formed by different C. bifermentans strains. Some B. cereus strains form short pilus-like appendages and others form sword-like appendages. Spores also have variability in appendage anchorage. For example, some spores may be attached to the spore coat, as in C. botulinum type E while some spores emanate from the exosporium, as in some B. cereus strains. Accordingly, compositions of the disclosure such as spore display systems, fusion proteins, vaccines and other compositions described above may comprise a polypeptide or protein of interest displayed with or one or more spore proteins or spore peptides that are typically expressed on the surface of a spore.

In some embodiments, the present disclosure describes compositions and methods comprising Clostridium taeniosporum spores. Clostridium taeniosporum forms unique endospores having about twelve large, flat, ribbon-like appendages attached through a common trunk at one end of the spore. The present inventors have characterized Clostridium taeniosporum appendages and found them to be very large, approximately 0.5 microns in width, 4.5 microns in length, and 30 nanometers thick, and comprising three major structural proteins, one of which is a glycosylated protein (glycoprotein) GP85 and a mixture of two paralogous, highly similar proteins called P29a and P29b. The appendages may also comprise a fourth appendage protein, an ortholog of the Bacillus subtilis 26-residue spore morphogenetic protein SpoVM.

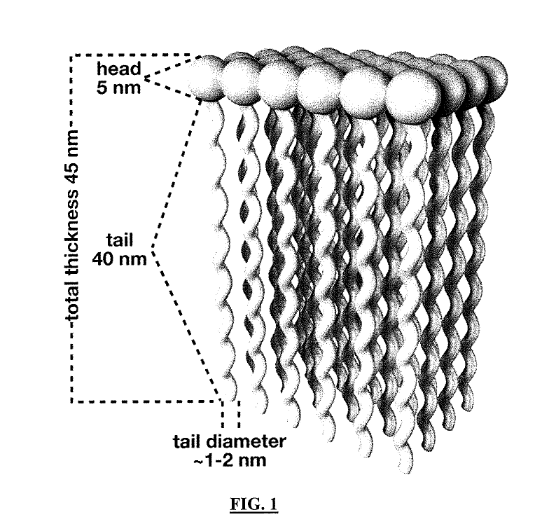

The present inventors have also characterized appendage ultrastructure of Clostridium taeniosporum spores by electron microscopy and atomic force microscopy (details described in the section entitled Examples) and found that Clostridium taeniosporum appendages are assembled to form small, tennis-racket shaped complexes with 5 nm diameter heads attached to long, thin tails about 40 nm long that are 1-2 nm in diameter as shown in the three dimensional model in FIG. 1. These small complexes assemble with the heads arranged in parallel rows to form one appendage surface and the tails extend away and form the second surface (FIG. 1).

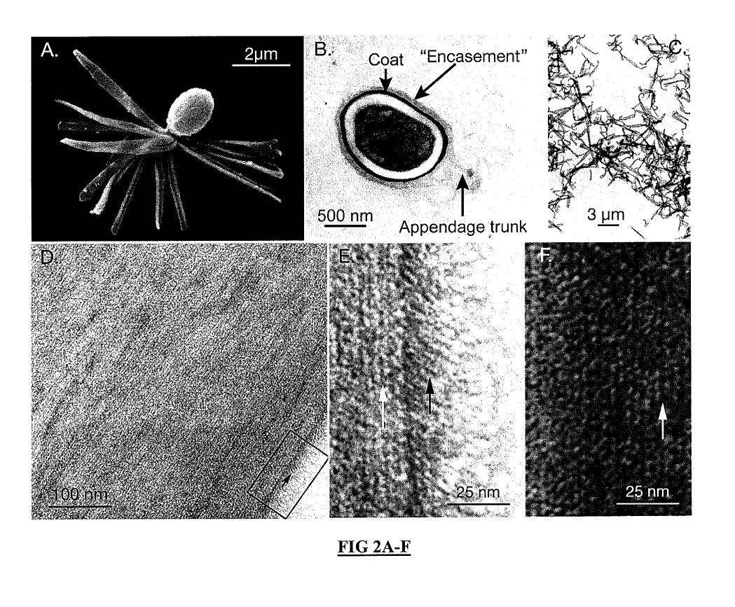

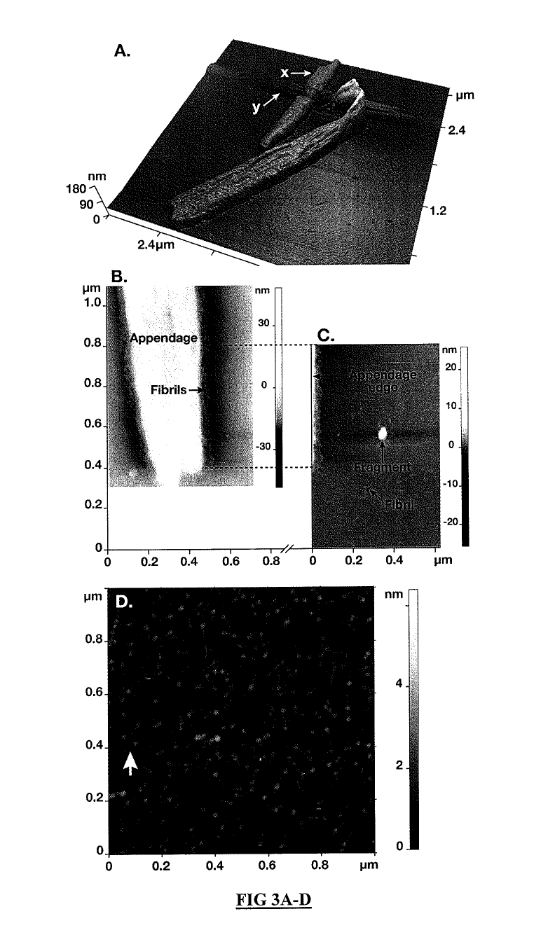

Electron micrographs of Clostridium taeniosporum appendages are illustrated in FIGS. 2A-2F, wherein FIGS. 2D, 2E, and 2F demonstrate an appendage surface formed by parallel rows of the bead-like small complex heads and atomic force microscopy images such as in FIG. 3D demonstrates the small tennis-racket-like complexes. Clostridium taeniosporum appendage heads may comprise two molecules of P29a and/or P29b and the tails may comprise one or two molecules of the collagen-like GP85. Collagen and collagen-like fragments may assemble into right-handed helices as shown in FIG. 1. Further details describing the experiments and analysis of appendage structures are provided ahead in the section entitled Examples.

The present inventors have also cloned and sequenced genes encoding the major appendage proteins: i.e., the nucleic acid sequence of the glycoprotein GP85 gene is described as SEQ ID NO. 1 and the amino acid sequence of the GP85 protein is described by SEQ ID NO. 2; the nucleic acid sequence of the non-glycosylated P29a gene is described by SEQ ID NO. 3 and the amino acid sequence of the P29a protein is described by SEQ ID NO. 4; and the nucleic acid sequence of the non-glycosylated P29b gene is described by SEQ ID NO. 5 and the amino acid sequence of the P29b protein is described by SEQ ID NO. 6.

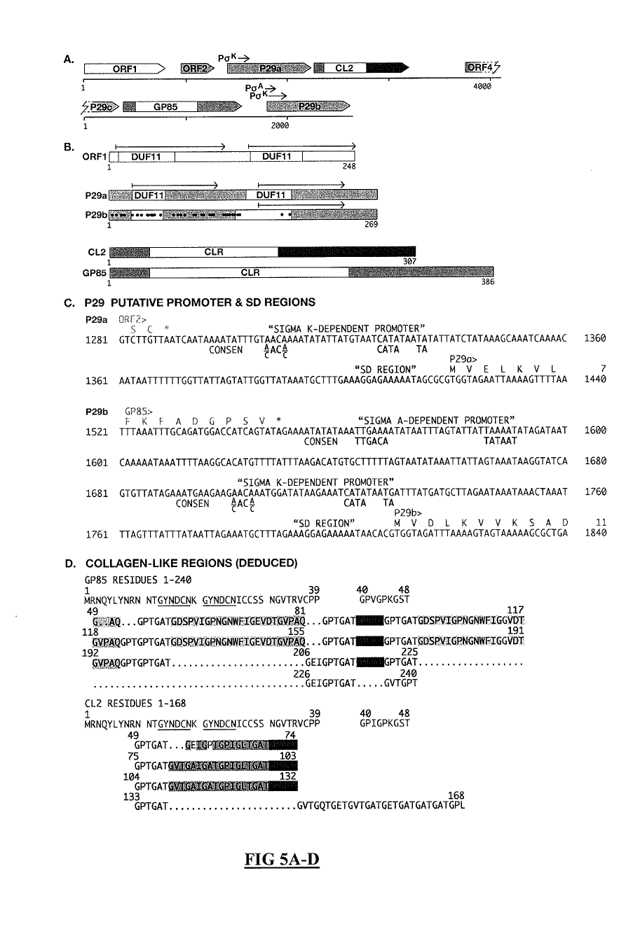

As shown in FIGS. 5A-5D, Clostridium taeniosporum P29a and P29b proteins comprise duplicated regions having a domain of unknown function 11 (DUF 11) and the GP85 glycoprotein contains a collagen-like region. The genes for P29a, P29b, GP85 and possibly related proteins are closely linked on two small chromosome fragments.

Some technical advantages of embodiments of the disclosure that relate to Clostridium taeniosporum spores are that their appendages may be easily broken from the spores and isolated, the appendages are very large, easy to isolate, easy to purify, easy to genetically manipulate, comprise one major glycosylated protein, and are non-toxic, making the appendages useful for several applications.

In one embodiment, the present disclosure describes a spore surface display system comprising a Clostridium taeniosporum spore configured (e.g., engineered) to have at least one nucleic acid encoding a polypeptide of interest, the spore further operable to express (e.g., transcribe, translate, translocate and/or assemble) the polypeptide of interest on the surface of the spore. In some embodiments, the spore may be operable to express a protein of interest on the spore surface by co-expression with a spore surface protein (i.e., carrier protein or peptide) such as but not limited to a GP85 protein, a P29a protein, a P29b protein and/or an ortholog of SpoVM, and peptide fragments or combinations thereof.

In some embodiments, the present disclosure describes a spore surface display system comprising Clostriduim taeniosporum spore transformed with a plasmid, which possesses a spore promoter operably linked to one or more nucleic acids encoding a polypeptide and at least one carrier, so that the spore promoter is operable to generate a fusion protein comprising the polypeptide and the carrier protein to be expressed on the surface of the spore. The carrier protein may be a spore coat protein, an exosporial protein, or a spore appendage protein. The plasmid has the ability to stably exist in the spore or integrate into the genome of the spore. In one embodiment, the spore promoter is preferably a mother cell promoter, more preferably, a late mother cell promoter.

In one embodiment, the plasmid employed to transform the spore may contain a general transcription promoter, which is capable of directing the expression of the fusion protein comprising the carrier protein and the polypeptide of interest in E. coli, or other suitable hosts. A technical advantage of having two promoters, one general transcription promoter and one spore promoter, in the plasmid, may be to allow the optimization and engineering the plasmid comprising the fusion spore appendage genes in a host, such as E. coli and then shuttle the plasmid into a spore, such as a Clostriduim taeniosporum spore.

In a non-limiting example, the plasmids comprising the fusion spore appendage genes may be engineered to introduce mutations into the carrier genes, such as appendage genes. The mutations may comprise insertion of epitope genes at different locations of the appendage genes. By expressing the engineered plasmids in E. coli and using appropriate screening method, the optimal insertion location of the epitope, may be identified and the corresponding plasmids are subject to subsequent transformation into a spore.

In some embodiments, the appropriate screening method referred to above is Anchored Periplasmic Expression (APEx).

APEx technique is based on anchoring a library of candidate binding proteins, such as antibody fragments, on the periplasmic face of the inner membrane of E. coli, or other suitable Gram-negative bacteria, followed by disruption of the outer membrane, incubation with fluorescently labeled target and sorting of the spheroplasts by flow cytometry (U.S. Pat. No. 7,094,571, incorporated by reference in its entirety). APEx may be used to screen for and identify biomolecules of interest (e.g., drugs, antibodies, antigens, chemicals, analytes, peptides, proteins, hormones, enzymes, substrates). In a non-limiting example, APEx may be used to isolate an antibody or an epitope which has a high affinity for a ligand. In one study, over 120-fold affinity improvement was obtained after only two rounds of APEx.

In a non-limiting example, the APEx technique may comprise the steps of 1) providing a Gram negative bacterium comprising an inner and an outer membrane and a periplasm, said bacterium expressing a nucleic acid sequence encoding a candidate binding polypeptide, wherein the candidate binding polypeptide is exposed within the periplasm of said bacterium; 2) contacting the bacterium with a labeled ligand under conditions wherein the labeled ligand contacts the binding polypeptide; and 3) selecting said bacterium based on the presence of said labeled ligand bound to said candidate binding polypeptide.

The APEx technique may be used to isolate affinity enhanced appendages from libraries of random insertion mutants. First, an inducible polycistronic vector in which appendage proteins will be co-expressed as either inner membrane associated or free in the periplasm in E. coli. or other suitable hosts, will be constructed. For example, the polycistronic vector may be IPTG-inducible. Then, an epitope gene of interest will be randomly inserted into an appendage protein gene by using a random insertion technique known in the art, such as insertional scanner linker mutagenesis (SLM) by using a modified Tn5 transposon. Following random insertion, insertional libraries of appendage protein genes, such as P29a/b and GP85 will be generated by individual cloning into the APEx polycistronic vector. Next, E. coli or other suitable hosts will be transformed with the insertional library, converted to spheroplasts, fluorescently labeled with an anti-epitope antibody, and sorted on a flow cytometer. Multiple rounds of sorting may be performed. Individual clone may be rescued by PCR amplification and subject to sequencing. The identified high affinity epitope insertion plasmid will then be transformed into C. taeniosporum.

The use of APEx technique in combination with the spore surface display system may offer one or more of the following advantages. First, in the absence of this high throughput screening method of APEx, the choice of which appendage proteins to mutate and the optimal position for the mutation, such as the insertion mutation can only be predicted by structural analysis of appendage proteins. Second, by using APEx, the best place to insert epitope into the appendage protein can be identified. For example, a well characterized epitope, such as FLAG, can be used in APEx analysis to identify the optimal insertion position. The identified position can be used to insert other epitopes of interest, such as a Shiga-like toxin 2 epitope. By inserting an epitope into the identified optimal position, the fusion protein displayed on the surface of a spore will have a higher affinity to a target molecule, such as a ligand or an antibody. The section entitled Example 7 provides a method for using APEx to demonstrate the technical advantages described above.

A technical advantage of some embodiments may be expression of numerous copies of a protein or polypeptide of interest using a Clostridium taeniosporum spore surface display system using an appendage protein as a carried since the numbers of carrier protein or polypeptide molecules per appendage are about 50,000 small complexes per appendage and/or about 600,000 such complexes per spore. Accordingly, in some embodiments, Clostridium taeniosporum spore derived compositions of the disclosure may be useful as biotechnological tools. For example, expression of a protein along with the numerous copies of carrier proteins may result in a uniform array of one or more multi-copy proteins or a repeated array of at least one protein. Such an array may be used as a biotechnology tool, some non-limiting example compositions may include an array of an enzyme, an array of a diagnostic protein, an array of a marker protein, an array of an antigen or an antibody.

In some embodiments, a nucleic acid encoding a polypeptide of interest may be cloned into an operon encoding a carrier protein in a spore surface display system comprising a Clostridium taeniosporum spore. In one embodiment, a nucleic acid encoding a protein of interest may be under the control of one or more spore protein promoters. For example, in a non-limiting example, a nucleic acid encoding a protein of interest may be under the control of a promoter or gene control sequence of a gene encoding for a carrier protein such as an spore appendage protein, such as but not limited to a GP85 protein, a P29a protein, a P29b protein and/or an ortholog of SpoVM.

The promoter and other regions controlling the open reading frames of these major appendage proteins have also been cloned, sequenced and characterized by the present inventors. For example, the control sequences for some Clostridium taeniosporum appendage proteins are depicted in FIGS. 5A-D, wherein the control sequence showing the putative promoter for P29a is represented by SEQ ID NO. 8, and the control sequence showing putative promoters for P29b is represented by SEQ ID NO. 9.

In one embodiment, the presence of a putative σ^{K}-dependent promoter sequences upstream of the P29a and P29b genes (see FIG. 5C) may indicate the translation of these genes at late stages in a mother cell of the spore. Without being bound to any theory, this may indicate expression of these appendage proteins late in the mother cell which is also consistent with their deposition into the layer external to the spore coat.

Spore display systems of the disclosure may be used to display any polypeptide of interest including, but not limited to, small polypeptides, larger proteins, epitopes, antigens, antibodies, hormones, enzymes, anticancer proteins, therapeutic proteins or peptides, and/or peptides or proteins that have an affinity for another biomolecule, toxic ions or molecules.

In some embodiments, the disclosure describes compositions comprising a spore expressing a polypeptide of interest on its surface such as but not limited to a spore surface display system, a fusion protein, a drug delivery device, a vaccine, a system for generating an antibody to an antigen/peptide expressed on a spore surface, an anticancer drug, an immobilized enzyme system, a system for serological reagent preparation, a contaminant removal system, a biocatalysis system, a screening platform, a nanotechnology platform, and/or a nanotechnology/biotechnology tool such as a bioanalytical sensor, a molecular electronic system and/or a signal processing system.

In some embodiments, the disclosure describes compositions comprising polypeptides expressed by a spore display system and may comprise a fusion protein, a protein that is expressed on the surface of a spore and/or optionally isolated therefrom.

In some embodiments, a spore display system of the disclosure may express a polypeptide of interest as a fusion protein. A fusion protein may comprise a polypeptide or protein of interest and a spore protein or spore peptide that is expressed on the surface of a spore.

In some embodiments, a composition of the disclosure may further be formulated as a pharmaceutical formulation that may be suitable for administration to a subject. In some embodiments, pharmaceutical compositions of the disclosure may also comprise adjuvants, other immunoactive agents, other therapeutic agents, pharmaceutical carriers and/or solvents. Pharmaceutical formulations are described in sections below.

Some embodiments of the disclosure relate to methods of making spore surface display compositions and/or other compositions as set forth above. A method according to the disclosure may include screening clostridial and/or bacillary plasmids or other Gram-Positive replicons for the ability to transform C. taeniosporum, screening different transformation (both chemically treated cells and cells exposed to electroporation) procedures, and different culture media and other reagents. One embodiment may include creating a plasmid predicted to transform and to be stable in C. taeniosporum by using a mini-chromosome, the replication of which is based on the C. taeniosporum origin of replication.

The disclosure, in some embodiments, describes methods for making fusion proteins.

In some embodiments, the present disclosure describes methods for generating proteins. As the spore based compositions of the disclosure produce high copies of proteins (for example, as described with Clostridium taeniosporum appendage proteins as carrier proteins) a system of the disclosure may be used to generate large quantities of protein. In some embodiments, methods may further comprise isolating proteins. As proteins are expressed on the surface of spores, isolation of protein may involve fewer steps and contaminants thereby may produce substantially pure isolated proteins.

In some embodiments, the disclosure describes methods of screening for and identifying biomolecules of interest (e.g., drugs, antibodies, antigens, chemicals, analytes, peptides, proteins, hormones, enzymes, substrates) using a composition of the disclosure. A high throughput screening method of the disclosure may comprise: 1) expressing a protein or peptide of interest that is operable to selectively bind to a biomolecule of interest on a spore surface display system of the disclosure; 2) contacting the spore surface display system with a sample having or suspected of having the biomolecule of interest; and 3) identifying binding of the biomolecule of interest to a peptide of interest on the spore surface display system.

In some embodiments, an enzyme may be displayed on the surface of a spore and may be used as a biocatalyst system. Such a method may be used for catalyzing chemical or biological reactions and may circumvent the need for expression and isolation of a stable enzyme. In some embodiments, such a method may be used for degrading toxic biochemicals or chemical agents using an immobilized spore display system of the disclosure that expresses an enzyme operable to degrade a toxic agent. In some embodiments, such a method may be used for converting one biochemical or chemical agent to another using an immobilized spore display system of the disclosure that expresses an enzyme. One example embodiment method may involve: 1) expressing an enzyme of interest on a spore surface display system that is operable to catalyze a biochemical or chemical reaction of interest; 2) optionally immobilizing the surface display system onto a surface; 3) contacting the spore display system (or the immobilized enzyme on the spore surface display system) with a sample having or suspected of having the chemical or biochemical agent for which catalytic conversion is desired; and 3) detecting the catalytic conversion. The method may also involve providing optimal buffers, conditions (temperature, pH) and other agents required for the catalytic process.

In some embodiments, systems of the disclosure may be used for adsorption of contaminants, wherein one or more contaminant may selectively be bound or adsorbed to a protein/polypeptide on a surface display system of the disclosure.

The present disclosure also describes methods for making nanobiotechnology tools that may comprise arrays of one or more protein that may be used for applications such as bioanalytical sensors, molecular electronics and signal processing.

In some embodiments, although appendages of some spores may be large, they are comprised of relatively few proteins. Since spore appendages assemble spontaneously in vivo from small complexes with 5 nm bead-like heads and long thin tails (which also assemble spontaneously) orderly structures may be assembled in vitro having nanometer dimensions. A substantially uniform appendage assembly may allows the synthesis of multiple copies of large, regular protein structures which are useful in several nano-biotechnology applications such as those described above.

For example, in Clostridium taeniosporum appendages, heads may comprise two P29a proteins and tails may comprise one or more GP85 proteins. Over-expression of head protein genes, in a suitable host such as E. coli, may be used to generate and purify complex head proteins. Head proteins may be then subject to spontaneous assembly in vitro to form spherical heads. These structures may be further allowed to form supramolecular structures similar to C. taeniosporum appendages (however, without the tails) having repeating 5-nm diameter spherical subunits. In some embodiments, a repeating supramolecular structure may be modified for a variety of applications. For example, head proteins may be modified genetically or by chemical synthesis methods and used to assemble repeating supramolecular structures with bioadsorbants on their surface that may be operable for removal of toxic agents. Other applications may comprise constructing a biosensor by modifying a head protein (or any other appendage protein or spore protein) that may be assembled into a repeating supramolecular structure to display or co-express an enzyme, a hormone, an antigen or an antibody, or a receptor for use as a biosensor.

In some embodiments, compositions of the disclosure may be used for analyzing protein structure. For example, as seen in spore surface display systems of Clostridium taeniosporum, expression of multiple copies of a polypeptide of interest in uniform arrays with the numerous appendage proteins may result in a lattice of proteins that may be suitable for structural analysis, including cryoelectron microscopy and atomic force microscopy.

Some embodiments of the disclosure relate to therapeutic methods comprising administering to a patient or a subject in need a therapeutic spore surface display formulation of the disclosure. For example, a spore surface display composition may be used as a vaccine. In one embodiment, a vaccine composition of the disclosure may comprise at least one immunogenic epitope derived from an infectious agent or a disease causing agent to be expressed and displayed on the surface of a spore. A therapeutic formulation of a vaccine of the disclosure may be administered to a subject via a suitable delivery method. Details regarding pharmaceutical formulations and therapeutic delivery methods are provided in sections below. The section entitled Examples provides a prophetic method for developing a vaccine using teachings of the present disclosure.

Some embodiments of the disclosure relate to methods for delivering drugs using compositions of the disclosure. For example, compositions of the disclosure may be used to bind to and/or deliver drugs.

In some embodiments, a therapeutic method may include delivering a therapeutic protein or a therapeutic agent attached to a protein, wherein the protein is displayed on a composition of the disclosure.

Some embodiments describe therapeutic compositions and methods for treating tumors and cancers. Spore surface display compositions of the disclosure may be modified chemically or by bioengineering to make them operable to deliver anti-cancer drugs or antibodies to a tumor. For example, in some examples anticancer drugs (including chemotherapeutic agents, radioactive agents, anticancer proteins, peptides or antibodies) may be attached or associated with a display system of the disclosure. In some examples, an anticancer antibody or anticancer protein may be expressed on a spore display system of the disclosure.

In some examples, an anticancer drug or protein may be co-expressed and/or chemically added on a spore display system of a disclosure in addition to a cancer marker. Addition of a cancer/tumor marker may selectively target a modified spore to a tumor cell. In some embodiments, a glycoprotein component glycan of a spore as modified according to the disclosure may be modified to achieve specific binding to a tumor cell as glycoproteins are involved in surface recognition and binding.

A spore surface display system derived from an anaerobic bacterium such as but not limited to Clostridium taeniosporum may selectively geminate and colonize in the hypoxic environment of a tumor, thereby concentrating one or more therapeutic agents provided by the spore composition at the tumor. In one embodiment, a C. taeniosporum spore may be genetically engineered to secrete an anti-cancer agent such as Tumor Necrosis Factor (TNF) or to display TNF on an appendage surface.

Embodiments of the present disclosure provide several advantages over other cellular surface displays some of which have been outlined in sections above. A technical advantage of one embodiment is the large number of molecules of appendage peptides or proteins that are present in each appendage. As some embodiments use appendage peptides and proteins as carriers for expression of a polypeptide of interest, numerous copies of peptides of interest are produced in the present spore display system. A technical advantage of one embodiment may arise from the nature of spores and spore appendages being inert, the nature of an expressed peptide of interest may not interfere with growth of the host cell. Another technical advantage of the inert nature of a spore may be the possibility to express polypeptides of various sizes (including larger polypeptides of interest). A technical advantage of one embodiment may arise from glycosylated residues in appendage proteins which may allow suitable modifications of glycan residues to improve and increase specific binding and/or targeting of spore compositions of the disclosure to certain tissues or cells.

Pharmaceutical Formulations and Delivery—Pharmaceutical and/or drug delivery spore compositions of the present disclosure may be used as an active ingredient in pharmaceutical compositions in the manufacture of medicaments and for the treatment of humans and other animal subjects or patients and may be administered in accordance with conventional procedures. While it is possible for a therapeutic spore composition of the disclosure to be administered alone, it may be preferable to present it as a pharmaceutical formulation comprising at least one active ingredient (e.g., a spore surface display composition comprising a nucleic acid encoding a therapeutic protein or a peptide, wherein the therapeutic protein or peptide is displayed on the spore; or a spore surface display composition comprising a displayed protein or peptide that is associated or bound with a therapeutic agent) together with one or more pharmaceutically acceptable carriers therefore and optionally one or more other active therapeutic agents.

According to some embodiments, a carrier may be acceptable in the sense of being compatible with the other ingredients of the formulation and not injurious to the mammal. In some embodiments, a therapeutic spore surface display composition of the disclosure may be derived from a non-pathogenic bacterium or fungi to avoid pathogenic effects to the mammal. Pharmaceutical compositions of spore surface display compositions of the disclosure may comprise combinations of therapeutic agents and a carrier, inert or active, making the composition suitable for diagnostic or therapeutic use in vitro, in vivo or ex vivo.

Immunological applications, wherein a spore display composition is used as a vaccine displaying an antigenic epitope on its surface may include agents such as adjuvants such as complete Freund's adjuvant (a non-specific stimulator of the immune response containing killed Mycobacterium tuberculosis), incomplete Freund's adjuvants, alum, and/or aluminum hydroxide adjuvant. One technical advantage of using the spore display system in immunological applications may arise from one property of spores and spore appendages that they only induce very weak innate immune responses. Such property confers the spore display system the ability to provide a neutral platform for developing a variety of immunological applications. Detailed discussion of this aspect is provided in the Example section below.

A pharmaceutically acceptable carrier may encompasses any of the standard pharmaceutical carriers, such as a phosphate buffered saline solution, water, and emulsions, such as an oil/water or water/oil emulsion, and various types of wetting agents. The compositions also may include stabilizers and preservatives. For examples of carriers, stabilizers and adjuvants, see Martin, REMINGTON's PHARM. SCI., 15th Ed. (Mack Publ. Co., Easton (1975)), incorporated by reference herein.

An effective amount may be an amount sufficient to effect beneficial or desired results. An effective amount or a therapeutically effective amount may be administered in one or more administrations, applications or dosages. A pharmaceutical composition of the disclosure may be administered to a mammal such as a human patient in need thereof. Mammals may include, but are not limited to, humans, murines, simians, farm animals, sport animals, and pets.

In some embodiments, the disclosure provides methods for preparing pharmaceutical formulations compatible for systemic delivery.

In some embodiments, the disclosure describes the introduction of targeting moieties (such as cancer markers) or modification of glycan residues on glycoproteins of the spore-derived compositions of the disclosure to provide cell, tissue or organ specific uptake, which may in some embodiments depend on the pathological status of the cell, tissue or organ (e.g., having a cancer that is hypoxic). Exemplary markers may include cancer markers, tissue specific cell membrane proteins and the like.

Methods of Administration—Administration or delivery of a pharmaceutical spore-derived therapeutic composition of the present disclosure may comprise any method which ultimately provides a therapeutic agent to cell/tissue/organ or site it is needed at. The spore-derived compositions of the present disclosure may be delivered to a patient by a variety of means, including, but not limited to, oral ingestion, sublingual administration, intranasal, intramuscular injection, subcutaneous injection, parenteral administration, intrabiliary or topical application.

In some embodiments parenteral administration may comprise intravenous administration, intraperitoneal administration, subcutaneous administration, intrathecal administration, injection to the spinal cord, intramuscular administration, intraarticular administration, portal vein injection, or intratumoral administration. Topical administration may include administration to skin, eye, or any mucosal membranes.

In some embodiments, a pharmaceutical composition of the disclosure may be contacted with a target tissue by direct application of the composition to the tissue. The spore-derived compositions, including drug delivery vehicles, vaccines, anticancer drugs, may be introduced into a patient in an amount sufficient to produce a desired clinical effect, including, but not limited to, effecting apoptosis of a cancer or a tumor cell, eliciting an immune response, effecting a change in gene expression (e.g., increase in expression, decrease in expression or silencing of a gene), a change in gene transcription, a change in translation, a change in protein structure, a post-translational modification of a protein, or the treatment and/or prevention and/or alleviation and/or amelioration of one or more symptoms or a medical condition.

Administration in vivo may be effected in one dose, continuously or intermittently throughout the course of treatment. During the initial determination of dosage requirements, monitoring parameters that define the condition/disease may be advisable. Methods of determining the most effective means and dosage of administration are known to those of skill in the art, in light of this disclosure, and may vary with the composition used for therapy, the purpose of the therapy, the target cell being treated, and the mammal being treated. Single or multiple administrations may be carried out with the dose level and pattern being selected by the treating physician. Suitable dosage formulations and methods of administering the pharmaceutical compositions may be empirically determined by those of skill in the art in light of this disclosure.

### EXAMPLES

The following additional examples are offered to illustrate some embodiments of the disclosure, and should not be viewed as limiting the scope of the disclosure.

### Example 1

### Appendage Ultrastructure

Spore and Appendage Preparation: C. taeniosporum 1/k was grown anaerobically at 30° C. on Brain Heart Infusion medium (Becton Dickinson) supplemented with 0.5% sodium thioglycolate and 2% agar from spores lyophilized by Rode et al. (1967). Cultures were grown in anaerobic jars containing AnaeroGen atmosphere generation system sachets (Oxoid), but remained viable if exposed to oxygen for periods up to 20 min. To allow spore formation, cultures were incubated 7-10 days, at which time about 90% of the cells had formed endospores.

Spores were purified extensively as described by Rode et al. (1967). The spore-cell mixture from 10 plates was collected in 21 ml distilled water and dispersed by mild sonication (30 sec with a Heat Systems-Ultrasonics model W185 Sonifier fitted with a macrotip, which did not break appendages or spores) and purified by discontinuous sucrose gradient centrifugation. Seven ml portions of the spore-cell mixture were layered on gradients (13 ml of 10%, 13 ml of 20%, and 17 ml of 40% sucrose) and centrifuged at 810×g for 15 min at 4° C. The cells and debris remained in the 10 and 20% layers. The 40% layers containing the spores were combined, the spores washed three times by centrifuging at 3,000×g for 10 min at 4° C. and resuspending in water. After the last centrifugation, they were resuspended in 0.069 M pH 7.8 phosphate buffer. Streptomyces griseus protease (Sigma) was added at a final concentration of 0.1 mg/ml and incubated at 37° C. for 4-6 hrs. The protease-treated spores were again washed three times in water (as above) and stored in 2.5 ml water in sealed containers (exposed to air) at 4° C.

Appendages were broken from spores by sonication and extensively purified on sucrose gradients. Chilled spores were sonicated with the same sonifier with a microtip for a total of 15 min (30 sec of sonication followed by 30 sec of cooling on ice). The sonicated preparations were purified on discontinuous sucrose gradients. 2.25 ml of sonicated spore-appendage mixtures were layered on gradients (5 ml of 10%, 8 ml of 20%, and 10 ml of 40% sucrose) and centrifuged at 600×g for 20 min at 4° C. The appendages remained in the top 7 ml and the spores sedimented into the 40% layer. The appendages were recovered from the uppermost 7 ml by centrifuging at 7,700×g for 15 min at 4° C., resuspended in phosphate buffer and treated with protease as above. They were washed three times by centrifuging and resuspending in water and stored at −20° C. The preparations contained only intact appendages and some appendage fragments, but no cells, spores or cell debris was detectable by TEM (FIG. 2C).

Spores from which appendages had been broken were recovered from the 40% layer, washed and stored under the same conditions used for spores containing appendages.

Electron Microscopy: Intact spores with appendages attached were examined by scanning electron microscopy (SEM). The spores were fixed by treatment for 10 min in 1% glutaraldehyde and 1% paraformaldehyde in 1-piperazineethane sulfonic acid, 4-(2-hydroxyethyl)-monosodium salt buffer, pH 7.2 (HEPES) buffer at room temperature, washed three times with HEPES buffer, dehydrated by passage through ethanol solutions of 10, 25, and 50% in water (20 min each), and two final exchanges in 100% electron microscopy grade acetone. Each exchange was done by forcing the fluid through a 0.3 μm Millipore filter held in a filter holder between two syringes. The filter holder was transferred through liquid carbon dioxide for critical point drying in carbon dioxide in a Samdri critical point dryer and the filter mounted on SEM stubs with carbon tab. The spores were given an approximately 10 nm sputter coat of 60/40 gold/palladium alloy in argon plasma at 2.5 kv and 20 mA for 30 sec at 5 cm distance from the target. Secondary electrons were imaged at 12 kv by a Philips 515 SEM at a resolution of 512×512 pixels on a retrofitted SEMICAPS digitizer. Images were processed and colorized with Adobe Photoshop (7.0).

Purified appendages were examined by transmission electron microscopy (TEM). A sample was diluted 1:100 in water, adsorbed onto plasma-discharged carbon film on a #300 copper grid by placing the grid on one drop of diluted appendages for 1 min, washed twice with deionized water by grid transfer, and negatively stained by transferring the grid twice to drops of 2% uranyl acetate in water. The grid was dried and the appendages imaged at 80 kv with a 1 Mb Advantage HR camera (Advance Microscopy Techniques, Danvers, Mass.) (http://www.amtimaging.com) on a Philips EM208 TEM. Periodicity of appendage beads was determined with the program NIH Image (http://rsb.info.nih.gov/nih-image/about.html).

Spores from which the appendage had been broken were fixed, negatively stained and sectioned for TEM. They were fixed with glutaraldehyde and paraformadehyde and washed with HEPES as for SEM, transferred twice to 10 and 20% glycerol in water, and suspended in 30% glycerol in water. Each fluid change was done by centrifuging at 6,000 rpm for 3 min and resuspending the pellet in the next solution. A small portion of a spore pellet centrifuged from the 30% glycerol was frozen on the face of a Leica Bullseye cryo-mounting pin in a Leica CPC cryoworkstation and plunged (100 m/sec) into a liquid propane bath cooled by liquid nitrogen to −155° C. (http://www.leica-microsystems.com). The frozen spores were transferred to the −90° C. chamber of a Leica Automatic Freeze-Substitution system, incubated in uranyl acetate in methanol for 7 days, and transferred to Lowicryl HM20 epoxy for polymerization with UV at −45° C. The polymerized preparation was ultrasectioned at 50-70 nm thickness with a Diatome 45° diamond knife with a Leica Ultracut ultramicrotome and examined without post-staining by TEM as indicated above.

Ultrastructure Observations: C. taeniosporum spores have about twelve, long, flat ribbon-like appendages attached to one spore pole as shown in FIG. 2A. As observed by transmission electron microscopy (TEM) of sections of spores from which the appendages had been broken, the appendages are attached through a common trunk to a previously undescribed spore surface layer that fits tightly around the coat as illustrated in FIG. 2B. This surface layer (about 60 nm thick) is different from exosporia, which are thin, loosely-fitting, sac-like enclosures (19) and from the coat, which is the electron-dense layer of FIG. 2B. This layer that extends into the appendages is referred to herein as an “encasement”. Spore and appendage component dimensions are described in Table 1.

TABLE 1

| | | Dimension | S.D.^{b} | n^{c} |
|---|---|---|---|---|
| A. | Spore Component (TEM)^{a} | | | |
| | Core diameter | 900 nm | — | — |
| | Cortex thickness | 100 nm | — | — |
| | Coat thickness | 40 nm | — | — |
| | Encasement thickness | 60 nm | — | — |
| B. | Intact Appendage | | | |
| | Length (TEM) | 4.5 μm | 0.7 μm | 65 |
| | Width (TEM) | 0.46 μm | 0.8 μm | 110 |
| | Periodicity of beads (TEM) | | | |
| | across the appendage | 4.6 nm | 1.8 nm | 65 |
| | along the appendage | 5.6 nm | 3.7 nm | 58 |
| | Bead diameter^{d} (average) (TEM) | 5.1 nm | | |
| | Thickness (AFM) | | | |
| | mean | 32.5 nm | 2.5 nm | 551 |
| | at ridges | 35 nm | 1 nm | — |
| | at furrows | 31 nm | 2 nm | — |
| C. | Fibril (AFM) | | | |
| | Length (Head plus tail) | 45 nm | 6 nm | 139 |
| | Head diameter^{e} | 5.2 nm | 0.2 nm | 150 |
| | Tail diameter | 1-2 nm | — | — |

^{a}Microscopy technique is indicated by parentheses.
Dashes indicate that no value was determined.
^{b}Standard deviation.
^{c}Number of measurements.
^{d}Calculated by averaging the bead periodicity along and across the appendages.
^{e}Calculated by the Henderson et al. (1996) equation. See Experimental Procedures.

FIGS. 2A-2F describe SEM and TEM micrographs of C. taeniosporum spore and appendage structures. FIG. 2A shows an SEM of spore with ribbon-like appendages where the spores were fixed in glutaraldehyde/paraformaldehyde in HEPES buffer, dehydrated in acetone, transferred through liquid carbon dioxide for critical point drying, and coated with gold/palladium alloy (see Experimental Procedures above). The images were colored and the background blackened by Photoshop.

FIG. 2B shows a TEM of a negatively-stained longitudinal section of a C. taeniosporum spore from which the appendages had been sheared. The spores were fixed as for SEM but transferred into glycerol/water. A portion of a pellet centrifuged from the glycerol/water was frozen on a cryo-mounting pin and plunged into liquid nitrogen. The frozen spores were negatively stained by uranyl acetate at −90° C. for 7 days, transferred into Lowicryl HM20 epoxy for polymerization, and 50-70 nm sections prepared and examined without post-staining (see Experimental Procedures). The coat (electron-dense layer) is surrounded by an outer layer, proposed to be called the encasement, which ended in a common trunk from which appendages were sheared.

FIG. 2C shows a TEM of appendages. Purified appendages were negatively stained with uranyl acetate, dried and imaged (see Experimental Procedures).

FIG. 2D shows a higher magnification of a portion of one appendage showing the surface and the edge. The surface consists of parallel rows of beads. Fibrils, indicated by the arrow, are visible as a hair-like nap on the appendage edge. The box indicates the region shown at higher magnification in FIG. 2E.

FIG. 2E shows a higher magnification of the appendage edge and fibrils from FIG. 2D. A row of beads is indicated by the white arrow; the fibrils by the black arrow.

FIG. 2F shows an additional TEM view of parallel rows of beads which form an appendage surface. The arrow indicates one row of beads.

Appendages, broken from spores and purified by sucrose gradient centrifugation, appear by TEM of negatively-stained preparations, as long, flat ribbon-like structures about 4.5×0.5μ with the attachment end tapered and bent into a semi-circle that is twisted relative to the ribbon (see for example FIG. 2C). A hair-like nap, about 40 nm wide and composed of thin fibrils, is visible along the edges (as in FIGS. 2D and 2E) which is similar to B. anthracia and B. cereus exosporia in the sense that both the appendages and exosporia have fibrils of about 40 nm length visible along the edges. The appendage surfaces appear as parallel rows of beads with a periodicity of about 4.6 nm across and about 5.6 nm along the appendage (FIGS. 2E, 2F), with 5.1 nm average bead diameter (Table 1).

Atomic Force Microscopy: Appendages (5 μl) were deposited on freshly cleaved mica or (1-(3-aminopropyl)silatrane)-treated mica and allowed to adsorb for 5 min, rinsed with a few drops of deionized water, dried in argon flow, and imaged at ambient conditions with a MultiMode SPM instrument equipped with an E-scanner as described or a Bioscope with a G-scanner (both instruments from Veeco, Santa Barbara, Calif.) operating in the Tapping Mode. High spring constant conical sharp silicon tips (Olympus, Japan) were used for imaging in air; tapping frequencies were 300-350 kHz and the scan rate was 0.7-1.5 Hz. Femtoscan Online version 1.6 (Moscow State University and Advanced Technologies Center, Moscow, Russia) was used for image analysis.

The molecular volume and corrected diameter of the fibril heads were calculated by approximating the head as a segment of a sphere using the equation:

$$V = \frac{π \times h}{6} \times ( 3 {r}^{2} + {h}^{2} ) ,$$

where V is the volume, h is the height, and r is the radius of the AFM images. This calculation is performed because AFM images of such small structures are know to appear wider and shorter than they are. The fibril head molecular weight was calculated from the volume assuming a partial specific volume of 0.73 cm³/g as is known in the art. The head diameter was calculated from the volume, assuming the heads are spherical (Table 1).

FIGS. 3A-3D show AFM results of appendage structure, wherein FIG. 3A shows approximately one-half of an appendage shown in three-dimensional view. The z axis is in nanometers (nm). The structure marked X is an appendage fragment. The groove labeled Y is an imaging artifact. FIG. 3B shows an appendage with fibrils forming a hair-like nap around the edges is shown in three dimensions. The color scale is the z axis. FIG. 3C shows three dimensional images of individual fibrils on the mica surface near the FIG. 3B appendage were made visible by expanding the z axis (the color scale). The appendage edge of FIG. 3B plus an additional area to the right of the appendage not shown in FIG. 3B are presented in this panel. The appendage edge (which appears white), a small fragment of an appendage with fibrils attached and some individual fibrils are indicated. FIG. 3D shows individual fibrils on the mica surface presented in three dimensions. The spherical heads are nearly white and the tails are long and thin. An arrow indicates several individual fibrils. The color scale is the z axis.

Atomic force microscopy (AFM) reveals that individual appendages are flat structures with a thickness to width ratio of about 0.1 (FIG. 3A). The average thickness is 32.5 nm but ranges from about 20 nm to about 40 nm near the attachment end. The appendage surface is rough with two to five ridges and furrows per appendage, but the number of ridges and furrows varies along the length of individual appendages. The bent attachment end extends about 170 nm and is naturally twisted relative to the ribbon (FIG. 3A). Fibrils were visible along the appendage edges. In FIG. 3B, height in nanometers (nm) is represented in brown scale. A flat ribbon about 30 nm in height, and which appears white, has hair-like fibrils visible along the edges. Small fragments of appendages with attached fibrils and individual fibrils, which apparently separated from the appendages during preparation or storage, were visible on the mica surface (FIG. 3C). These fibrils were about 45 nm in length including spherical heads about 5.2 nm in diameter and long, thin, 40-nm tails about 1-2 nm in diameter (FIG. 3D; Table 1).

### Example 2

### Appendage Composition

SDS-PAGE and staining: Purified native or deglycosylated appendages were mixed with an equal volume of 2× native buffer (0.125 M pH 6.8 TrisHCl, 20% glycerol, 10% dithiothreitol) and 0.25 volumes of 4× loading buffer, boiled for 3 min and electrophoresed on a 1 mm thick 8×10 cm 4-12% gradient NuPage gel (Invitrogen, Carlsbad, Calif.) according the manufacturer's protocol with 2-(N-morpholino)ethanesulfonate buffer (NuPAGE) at 200 v for 35 min. Gels were stained with Coomassie Blue. Glycoproteins were stained with the Periodic acid-Schiff (PAS) stain after electroblotting from the gel to 0.2 μpolyvinyldifluoride membranes (Applied Biosystems, Foster City, Calif.) in a Novex transfer apparatus (Carlsbad, Calif.). The blotting was at 24 v for 90 min.

Deglycosylation: 20 μl of appendages were lyophilized in 5 mm (outside diameter)×50 mm borosilicate glass tubes in a SpeedVac for 2 hr, mixed with 25 μl trifluoromethane sulfonic acid for 60 min at 0° C., and neutralized with 75 μl of 60% pyridine in water in a dry ice-methanol bath. Deglycosylated proteins were precipitated by methanol-chloroform. To 100 μl of sample, 400 μl of methanol, 300 μl of chloroform and 400 μl of water were added and mixed thoroughly and centrifuged at 9,000 rpm for 20 min at room temperature. The protein precipitated at the interface and the upper phase was carefully discarded. 300 μl of methanol were added and centrifuged as above. The liquid was discarded and the pelleted precipitate was dried in a SpeedVac and dissolved in native buffer.

N-terminal Peptide Sequencing: Automated Edman-based N-terminal protein sequencing was performed using a Procise 492 cic attached to a Model 140C Micro-gradient System and a 610A Data Analysis System (Applied Biosystems, Foster City, Calif.). Sequencing procedures were optimized in house according to the manufacturer's manual. Membrane bands containing proteins were cut out, cut into smaller pieces, and loaded into the reaction cartridge of the sequencer prior to starting the sequencer. Intact, insoluble appendages were also subjected to N-terminal sequencing. Appendages were centrifuged from 25 μl, dissolved in 25 μl of glacial acetic acid, diluted with 75 μl of 50% acetonitrile in water, and 2.5 μl were spotted on a glass fiber filter which was loaded into the reaction cartridge.

Mass Spectrometry: Bands of P29 and deglycosylated GP85 proteins were excised from gels, destained, and digested in-gel with trypsin (0.6 μg). Tryptic peptides were extracted, acidified in 5% formic acid and purified by reversed phase chromatography with POROS R2 (Applied Biosystems, Foster City, Calif.). Purified peptides were then analyzed by nanospray ESI-MS/MS using a QStar pulsar mass spectrometer (Applied Biosystems). Peptides were directly infused using nanospray emitters (PROXEON, Odense, Denmark) and spectra collected in full MS mode to determine parent masses and their charge states. Candidate ions were then subjected to MS/MS and the collision energy adjusted for optimal fragmentation. The resultant MS/MS spectra were analyzed with BioAnalyst software (Applied Biosystems) to obtain partial amino acid sequences.

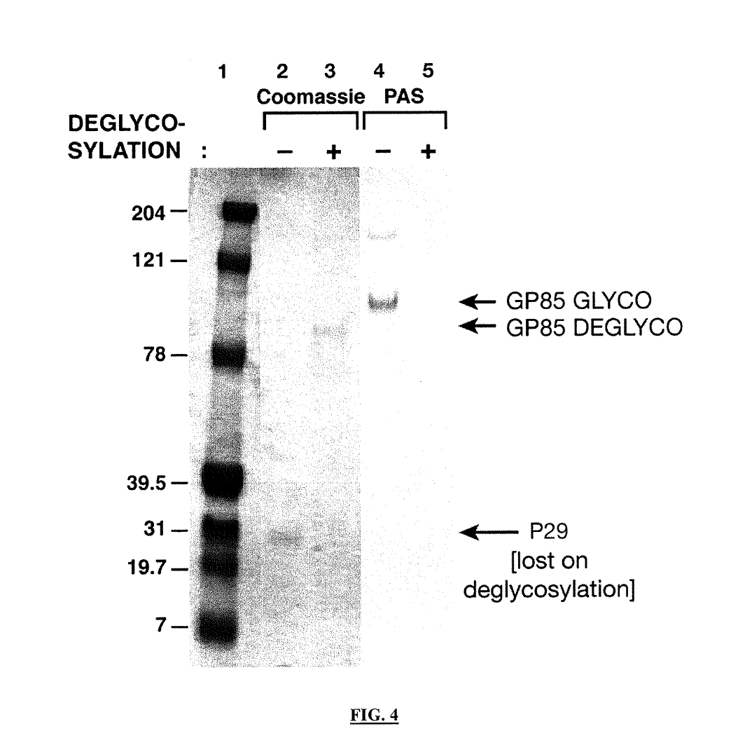

FIG. 4 shows an SDS-PAGE gel of appendage proteins, before and after deglycosylation, solubilized by boiling in dithiothreitol-containing buffer wherein, Lane 1: molecular weight markers; Lanes 2, 3: are stained with Coomassie Blue; and Lanes 4, 5: are stained with Periodic Acid-Schiff's (PAS) reagent for glycoproteins.

Appendage Composition: Four proteins, one of which is a glycoprotein, have been detected in purified appendages. Native appendages, boiled in buffer containing dithreithreitol and subjected to SDS-PAGE, yielded two bands, one of about 29 kDa visible by Coomassie Blue staining and a 100 kDa band visible only by glycoprotein staining (FIG. 4). As shown further below, the 29 kDa band consists of (at least) two isoforms designated P29a and P29b.

The glycoprotein migrated with an apparent molecular weight of 85 kDa after deglycosylation and is designated GP85. Glycoprotein staining sometimes detected small amounts of two additional proteins which migrated with apparent molecular weights of 155 and 135 kDa, but they were not observed in all preparations and were not studied further.

Evidence for the presence of a fourth protein, which may be an ortholog of the essential, 26-residue B. subtlis morphogenetic spore protein SpoVM is presented in sections below.

### Example 3

### DNA and Protein Analysis

Molecular techniques: Chromosomal DNA was isolated with the Puregene Genomic DNA Purification Kit (Gentra Systems, Minneapolis, Minn.). Plasmids were isolated with the Qiagen Plasmid Mini Kit, Qiagen, Inc. Valencia, Calif. PCR was done with the Expand High Fidelity Enzyme Mix, Roche Diagnostics Corp., Indianapolis, Ind. The pGEM-T Easy vector system was from Promega, Madison, Wis.

P29 and GP85 gene sequencing and analysis: A 487 bp fragment of the P29a gene was amplified from chromosomal DNA using the P29 F1 forward primer 5′ ATGGTWGAATTAAAAGTWTTA (wherein W is A and T) (SEQ. ID NO. 10) and P29 R7 reverse primer 5′ ATCATAAAAIACIACATTATCTA (SEQ. ID NO. 11). The sequence of the product was used to design primers for inverse PCR to amplify flanking regions of the chromosome which were also sequenced. This approach extended the sequence to 4101 bp. A 1.2 kbp fragment of the GP85 gene was amplified with GP85 F1 forward primer 5′ ATGAGIAATCAATATTT (SEQ. ID NO. 12) and reverse primer (R20) 5′ GTAGCAGCATCWSWATTACCAACTCC (wherein S is C and G) (SEQ. ID NO. 13). This region was also extended by inverse PCR and sequencing to 2.6 kbp. A PCR product of chromosomal DNA containing the P29a gene was amplified with primers P29 F6 and P29 R19 and cloned into the pGEM T-Easy vector (Promega, Madison, Wis.). The amplification conditions, enzymes used for inverse PCR circularization and the primers used for amplifying and sequencing both the 4.1 and 2.6 kbp fragments are available on the world wide web at sbs.utexas.edu/walker/cl.taeniosporum.spore.appendage.genes.

Protein sequences were compared and analyzed by BLAST (Altschul et al., 1997), BLAST2 (Tatusova and Madden, 1999), RADAR (Heger and Holm, 2000) and MYHITS (Falquet et al., 2002).

FIGS. 5A-D illustrate the organization of chromosomal regions containing genes for P29, GP85 and possibly related proteins. Numbers indicate nucleotide pairs. FIG. 5 A shows ORF positions and directions of transcription are indicated by arrows and Pσ^{A} and Pσ^{K} are putative sigma A- and K-dependent promoters, respectively. ORF 4 and P29c are incomplete.

FIG. 5B shows characteristics of ORF1, P29a and P29b, CL2 and GP85 proteins deduced from their DNA sequences. Numbers indicate amino acid residues. In ORF 1 and P29a and b, arrows indicate repeat regions and white bars indicate domains of unknown function 11 (DUF11). The black dots in P29b indicate residues different from those in P29a. In CL2 and GP85, white bars indicate collagen-like regions (CLR), the blue bars indicate 39 identical N-terminal residues and the black and purple bars indicate regions with no significant homology.

FIG. 5C shows putative promoter and SD regions upstream of the P29a and P29b genes. Black and green numbers indicate by and amino acid residues, respectively. C-termini of the ORF2 and GP85 genes are indicated in orange. N-termini of the P29a and P29b genes are indicated in green. Putative σ^{K}- and σ^{A}-dependent promoters are indicated in blue. Consensus sequences are indicated by CONSEN. A putative SD region is shown in red. Quotation marks around the putative promoters and SD regions indicate that their assignments are tentative.

FIG. 5D shows N-terminal 240 and 168 residues of GP85 and CL2 proteins. Collagen-like regions are residues 40-240 and 40-168 in GP85 and CL2, respectively. The dots indicate gaps. The N-terminal 39 residues (blue) are identical and contain the sequence GYNDCN repeated (underline) (SEQ ID NO. 14). Residues 40-48 are identical except for positions 42. Other repeat regions are indicated by different colors.

Sequences of chromosomal fragments containing genes for P29a, P29b and GP85 proteins: The amino acid sequences of the N-termini and of internal oligopeptides of the P29 and GP85 gel bands as shown in Table 2 were used to design primers for amplification and sequencing chromosomal fragments containing portions of the relevant genes. The initial fragments were then extended by inverse PCR and chromosomal regions of 4.1 and 2.6 kbp, the former containing the gene for P29a and the latter the genes for GP85 and P29b, were sequenced (FIG. 5A).

TABLE 2

| A. N-terminal sequences^{a} | | Present in |
|---|---|---|
| MVELKVLXSADRSYVFFGIXN | (SEQ ID NO. 26) | P29a P29b |
| MRNQYLXNRNNTG/TYND | (SEQ ID NO. 27) | GP85 |
| MKFYTNK | (SEQ ID NO. 28) | SpoVM |

B. Internal oligopeptides in P29a and b

| | Sequence | | Present in | |
|---|---|---|---|---|
| Number | (Corrected)^{b} | | P29a | P29b |
| P29-2 | LDNVVFYDSLPK | (SEQ ID NO. 29) | + | + |
| P29-4 | YSLSLTNIGDTK | (SEQ ID NO. 30) | + | − |
| P29-5 | VFFGLSNR | (SEQ ID NO. 31) | + | − |
| P29-14 | VSYSVLLTNNSNLK | (SEQ ID NO. 32) | + | + |
| P29-15 | VLTIPVIR | (SEQ ID NO. 33) | − | + |

C. Internal oligopeptide in GP85

| | | |
|---|---|---|
| GP85-2 | PPGPVGPK | (SEQ ID NO. 34) |

^{a}X represents an unknown residue; (G/T) could be G or T.
^{b}The P29 internal peptides 4, 5 and 14 were initially sequenced as YSLSLTTLSLP (SEQ ID NO: 35), VFFGLESS (SEQ ID NO: 36) and SYSVNLTNG (SEQ ID NO: 37) but corrected upon reexamination of the raw data in light of the deduced amino acid sequences of the P29 genes. Mass spectrometry could not distinguish I and L; the final assignments were based on the deduced
amino acid sequences.

P29a Protein Coding Region: From the P29 protein band, N-terminal and internal oligopeptide sequences were determined (see Table 2). A forward primer based on the first 7 residues of the N-terminal amino acid sequence and a reverse primer based on the first 8 residues of internal oligopeptide 2 amplified a 487 bp fragment of the chromosome. [Degenerate primers included the most frequently used codons (at least 50% of the total) in C. acetobutylicum, C. perfringens and C. tetani (http://cmr.tigr.org/tigr-scripts/CMR).] This fragment was a portion of a P29 gene, based on the fact that the deduced amino acid sequence of the encoded reading frame matched the P29 protein band N-terminal residues 9 through 21 and P29 internal oligopeptides 4, 5 and 14. N-terminal residues 8 and 20, uncertain from the Edman sequencing, were identified by the DNA sequencing. The 487 bp sequence of the P29 gene was used to design additional primers for inverse PCR, extending the sequence to 4101 bp which contain an entire P29 gene (designated P29a), three additional ORFs (ORF1, ORF2, and CL2) and a portion of ORF4 as shown in FIG. 5A.

The P29a gene is downstream of ORF2 by 129 bp in which is located a putative σ^{K}-dependent promoter as shown in FIGS. 5A and 5C, the sequence of which matches the consensus sequence. This is consistent with P29a expression late in the mother cell, based on analogy to function of the B. subtilis SigK and the assumption that C. taeniosporum also encodes SigK, as do the other clostridia for which data are available. The P29a gene also is preceded by sequences, centered around both the −15 and −9 positions, which are similar to Shine-Dalgarno (SD) sequences (see FIG. 5C).

ORF1, encodes a protein related to the P29a protein (FIGS. 5A, 5B), and ORF2 (FIG. 5A) product functions are unknown. The CL2 gene encodes a protein that includes a collagen-like domain, as does the GP85 protein (see below). Downstream of the CL2 ORF is the 5′ end of ORF4, a glycosyltransferase gene. The deduced 102 N-terminal residues are about 60% identical to those in similar regions of glycosyltransferases probably involved in cell envelope synthesis in the bacilli and clostridia. ORF4 protein might be involved in glycosylating GP85 or other spore components. The 4101-nucleotide sequence of this regions has been deposited with GenBank with Accession number DQ826675 (SEQ ID NO. 15).

Region Coding for GP85 and P29b Proteins: A forward primer, based on the GP85 band first six N-terminal residues, and a reverse primer based on an erroneously identified internal GP85 band oligopeptide nonetheless amplified a 1.2 kbp fragment of chromosomal DNA which was a portion of the GP85 gene. The deduced amino acid sequence of the encoded reading frame matched residues 8-16 of the GP85 gel band N-terminus and the GP85 internal oligopeptide 2. The 1.2 kbp fragment was extended by inverse PCR to 2.6 kbp which included the GP85 gene, a paralog of the P29a gene, designated P29b, and a portion of another potential P29 isoform gene, P29c (FIG. 5A).

GP85 protein consists of 386 residues with a deduced molecular weight of 37,498 (SEQ ID NO. 2). It includes a collagen-like region over residues 40-240 containing 53 GXY sequences and longer duplicated regions (FIG. 5B, 5D). The discrepancy between the deduced molecular weight of 37.5 kDa and the observed migration as an 85 kDa protein (after deglycosylation) in SDS-PAGE gels presumably results from the collagen-like region. Collagen monomers and fragments have been shown previously to migrate anomalously slowly in SDS-PAGE and the degree of retardation relative to the molecular weight was about the same as that observed with the GP85 protein.

The P29b protein is also an appendage component because mass spectrometric analysis of the P29 band from the gel detected the P29 internal oligopeptide 15 which is encoded by the P29b, but not the P29a, gene. The P29 gel band internal oligopeptide 14 is present in both P29a and b proteins (Table 2). The P29b ORF is separated from the preceding GP85 ORF by 259 nucleotide pairs which include putative σ^{A}- and σ^{K}-dependent promoters (FIG. 5A, 5C). The P29b potential SD region is identical to that upstream of P29a (FIG. 5C). Upstream of the GP85 ORF is the 3′ end of another potential P29 isoform gene, P29c. The deduced amino acid sequence over 114 residues is identical to the C-terminal 114 residues of P29a and 98% identical to a similar region of P29b. The sequence of the 2.6 kbp region has been deposited with GenBank with Accession number DQ832181 (SEQ ID NO. 16).

A Fourth Appendage Component: A fourth appendage protein was detected by Edman N-terminal sequencing of intact appendages. The first cycle identified only methionine, but each cycle 2 through 7 identified three residues. In each of those cycles, one of the amino acids was derived from the P29a protein and one from the GP85 protein. Detection of a third residue in each cycle indicated the presence of another protein, the sequence of which was KFYTNK (over residues 2-7) (SEQ ID NO. 7). Assuming that its N-terminus is M, the first 7 residues match 6 of the first 7 residues of SpoVM, a 26-residue morphogenetic protein of B. subtilis and other spore formers. The appendages may include a fourth protein which is an ortholog of SpoVM or of another protein with similar N-terminal sequence.

Spore Appendage Ultrastructure, Composition, Nucleic Acid and Protein Sequences: C. taeniosporum spores have about 12 large, ribbon-like appendages attached to a structure that surrounds the spore coat. Because this external layer is external to the coat, fits tightly around the spore, is extended through a common trunk at a specific spore pole into the appendages and is unlike loosely-fitting exosporia described earlier, accordingly this layer is referred to herein as an “encasement”.

As seen by TEM, appendage flat surfaces are composed of parallel rows of beads of 5.1 nm diameter with thin fibrils about 40 nm long visible along the appendage edge (FIGS. 2D, 2E, 2F). As seen by AFM, fibrils were also visible along the appendage edge and individual fibrils with spherical heads about 5.2 nm in diameter (Table 1) and long, thin 40 nm-tails could be seen on the mica surface. The individual fibril heads seen by AFM presumably are the beads seen by TEM on the appendage surface (they had almost identical diameters) and the fibril tails presumably form the hair-like nap seen by TEM along the appendage edges. The fibril tail may be formed by the collagenous domain of GP85. The head, with a molecular volume of about 58.5 kDa (see Experimental Procedures), may be formed primarily from one molecule each of P29a (29.3 kDa) and P29b (29.4 kDa), a total of 58.7 kDa.

The P29a and b and the GP85 genes, along with three other genes and portions of ORF4 and P29c, are located on two chromosomal regions totaling 6.7 kbp in length. All the genes on each fragment are transcribed in the same direction. All these genes may be involved in encasement, trunk and appendage synthesis or assembly because the deduced protein products share several common features, including sequence homology, conserved motifs and enrichment in cysteines. The shared motifs include the conserved domain of unknown function (DUF11) in P29a/b and ORF1 proteins and the collagenous domains in GP85 and CL2 proteins.

Analysis of P29 and ORF1 Protein (Deduced) Sequences: The P29a and b proteins are very similar, both with 269 residues, 74.6% identity over the N-terminal 130 residues, 98% identical over the C-terminal 139 residues (FIG. 5B) and molecular weights of 29,262 and 29,395. There are also duplicated sequences within each protein. In P29a, an 87-residue region in the N-terminal half is 26% identical and 47% similar to an 87-residue region in the C-terminal half and a domain of unknown function 11, DUF11, is present in both N and C terminal halves. The P29b protein also contains two DUF11 regions, although the one near the N-terminus is short and perhaps is only a DUF11 fragment.

The P29a/b proteins are related also to the ORF1 protein in primary sequence, in the presence of DUF11s, and in consisting of duplicated regions (FIG. 5B). First, P29a residues 8-236 are 29% identical and 47% similar to ORF1 residues 17-248 and there is significant homology also between P29b and ORF 1 proteins. Second, is the presence of extensive duplicated regions −87 and 111 residues in P29a and ORF1 proteins, respectively (FIG. 5B). Third, two DUF11 regions are present in all three proteins. The DUF11 motif is about 53 residues and often found in repeated regions of cell envelope proteins of unknown molecular function.

Examples of the proteins and the phylogenetically distant organisms which produce them include two large (over 220 kDa) conserved repeat domain proteins of unknown function of B. anthracis and a 25 kDa putative membrane protein of unknown function of Methanobacterium thermoautotrophicum. DUF11-containing proteins are major structural components of the outer membrane of the Chlamydia, organisms which do not contain peptidoglycan.

Repeat Regions in Structural Proteins: Some cell envelope structural proteins, the appendage structural protein P29a, and the possibly related ORF1 protein, have duplicated regions sometimes including DUF11s in both regions. Although the molecular function has not been determined for any of those proteins, they may be expected to have two very similar tertiary structures connected by a linker. Such repeat proteins may be suited for macrostructure assembly because single molecules would bind more molecules of surrounding proteins than would structural proteins that do not consist of duplicated structures.

Analysis of GP85 and CL2 Protein (Deduced) Sequences: The GP85 protein consists of 386 residues (SEQ ID NO. 2) with an internal 201-residue collagen-like region with 53 GXY triplets (FIG. 5D). The collagenous domain is organized into long repeats; residues 49-117 and 118-191 are almost identical, each containing three GPTGAT (SEQ ID NO. 17), one or two GVPAQ (SEQ ID NO. 18), and one GPCCT sequence (SEQ ID NO. 19) and two 19-mers GDSPVIGPNGNWFIG(E/G)VDT, (SEQ ID NO. 20) designated the GP85-motif. Residues 192-225 contain portions of the two longer repeats. The GPTGAT (SEQ ID NO. 17), GVPAQ (SEQ ID NO. 18) and GPCCT (SEQ ID NO. 19) sequences are repeated ten, four and three time, respectively within the collagenous domain.

The GP85 protein is related to the CL2 protein (SEQ ID NO. 21), which also contains a collagen-like region (FIG. 5A, 5B, 5D). The CL2 protein 129-residue collagen-like region is entirely GXY repeats, except for three GPCCT pentamers (SEQ ID NO. 19). The two proteins are also related in primary sequence. They are identical over the first 39 residues and the collagenous regions start at residue 40 in both proteins and 8 of the residues 40-48 are identical. The GP85 and CL2 proteins both contain repeats (FIG. 5B, 5D), including GYNDCN (SEQ ID NO. 14) twice in the 40 N-terminal residues and GPTGAT (SEQ ID NO. 17) and GPCCT (SEQ ID NO. 19) sequences in the collagenous domains.

As in the GP85 protein, the CL2 collagenous region is organized into long repeats; residues 75-103 and 104-132 are identical, each containing a GPTGAT (SEQ ID NO. 17), an 18-mer GVTGAIGATGPIGLTGAT (SEQ ID NO. 22), designated the CL2 motif; and a GPCCT (SEQ ID NO. 19). Residues 49-74 are very similar to these repeats. Downstream of the collagenous region, the GP85 and CL2 sequences are not significantly homologous. Although the GP85 and CL2 proteins share some common features, there is no evidence that CL2 is an appendage component. A collagen-like protein with 60 consecutive, centrally located GXY repeats is encoded by the C. perfringens clg gene, but its function is unknown.

Collagenous Domains of Exosporial Proteins: Proteins with collagen-like regions are major structural proteins in exosporia. The B. anthracis exosporium major component, and the immunodominant antigen of the spore surface proteins, is a glycoprotein with a collagen-like central region (BclA). The BclA protein of the Sterne strain 7702 contains 68 consecutive GXY repeats, including 6 copies of the 21-mer BclA motif. However, the BclA collagen-like region is extremely polymorphic in B. anthracis, varying from 17 to 91 GXY repeats among 12 strains. This variation in collagen-like region length is responsible for differing lengths of hair-like filaments on the exosporium surface, indicating that the BclA protein is a structural component of those filaments. Although there is no significant sequence homology of the BclA non-collagenous region to proteins of known structure, the structure of the C-terminal two-thirds of BclA resembles that of the C1 q human complement protein. Both interact with the lung alveolar surfactant layer, suggesting that BclA might be a virulence factor. BclA homologs are present also in the exosporia of other B. cereus group members (B. cereus E33L and B. thuringiensis 97-27; GenBank Accession numbers CP000001 and AE017355, 95095491.1 respectively) and in B. licheniformis (http://cmr.tigr.org/tigr-scripts/CMR/shared/AnnotationSearch.cgi).

SpoVM: A B. subtilis SpoVM ortholog (or another protein with an N-terminal sequence almost identical to that of SpoVM) was identified as an appendage component. SpoVM is a 26-residue spore morphogenetic protein that anchors the assembling coat to the outer forespore membrane by binding both the membrane lipid bilayer and the coat basement layer. Location of the C. taeniosporum SpoVM protein in appendages indicates a function different from that in B. subtilis and/or an additional function.

Cysteine Content: Another characteristic of the proteins encoded by the appendage genes and possibly related proteins is high cysteine content. ORF1, ORF2, P29a, CL2, GP85 and P29b proteins contain 2.8, 2.7, 2.6, 3.6, 2.8, and 3.0% cysteine, respectively, compared to 1.1-1.2% cysteines in total proteins (based on codon usage) among the three other clostridia for which data are available (http://cmr.tigr.org/tigr-scripts/CMR/shared/CodonUsage). This high concentration of cysteines might indicate that the appendage components are covalently linked by disulfide bonds, which is significant because bacterial and eukaryotic cytoplasm is a reducing environment. Disulfide bonds are commonly formed by disulfide oxidoreductases in the oxidizing environments of bacterial periplasm or non-cytosol compartments of higher organisms. For example, Chlamydiae elementary bodies lack peptidoglycan and the cysteine-rich major outer membrane proteins are crosslinked by disulfudes to maintain osmotic stability. The fact that a major B. anthracis exosporium constituent, the BclA protein, is also cysteine-rich raises the possibility that disulfide cross-linking may be involved in exosporium assembly also. If appendages and exosporia contain disulfides, are they formed in the mother cell cytoplasm before lysis or extracellularly after lysis.

Glycosylation: Another feature of the protein encoded by the appendage genes is glycosylation. Bacterial cells, adhesins, pili, flagella, S-layers and spore surfaces contain glycoproteins and both O- and N-linked glycosylation systems have been described. The observation that S-layer glycan deficient mutants arise during laboratory culture supports the idea that the glycans provide a selective advantage in natural environments. Glycoproteins also are components of other spore surfaces, including the BclA exosporium protein of B. anthracis.

### Example 4

### C. taeniosporum Pathogenicity

C. taeniosporum was described as a new species in 1968 and only one strain, designated 1/k, was described. Although this organism was the subject of early studies on appendage structure and synthesis, it was not included in the Approved List of Clostridium species compiled in the 1980s. Based on 16S rRNA homology, C. taeniosporum has been shown by the present inventors to share a common ancestor with the non-proteolytic C. botulinum types B, E, and F, and branched out earlier than those organisms.

Although closely related to these C. botulinum types, C. taeniosporum is not toxigenic. As set forth earlier, several therapeutic applications have been described for C. taeniosporum. Accordingly, toxicity analysis was performed.

Chromosomal DNA was extracted from exponentially growing C. taeniosporum cells by the Puregene Genomic DNA Purification Kit (Gentra Systems, Minneapolis, Minn.). A 1.46 kb portion of the 16S rDNA gene was amplified with the Expand High Fidelity Enzyme Mix (Roche Diagnostics, Indianapolis, Ind.) using generic clostridial rDNA primers (SEQ ID NO. 23 and SEQ ID NO. 24), purified by a QIAquick PCR Purification Kit [QIAGEN. Inc., Valencia, Calif.] and sequenced with an Applied Biosystems Model 3730 Automated DNA Sequencer. The sequence has been deposited in GenBank with the accession number EU696947 (SEQ ID NO. 25).

To test for the presence of botulinal neurotoxin and associated genes by PCR, total genomic DNA was isolated from C. taeniosporum and C. botulinum strains by lysozyme and proteinase K treatment. DNA was diluted to 50 ng/μl for PCR amplifications using the GeneAmp® High Fidelity PCR System (Applied BioSystems). PCR cycles were as follows: 95° C. for 2 minutes, followed by 25 cycles of 95° C. for 1 minute, an annealing step for 45 seconds at 48° C., 72° C. for extension, followed by 1 cycle of 72° C. extension for 10 minutes. Extension time depended on the length of the fragment being amplified. Genes tested for, and primers used, [25] are identified in Table 3.

Toxicity analysis was performed using a mouse bioassay to assess the toxicity of C. taeniosporum. Cultures grown for four days in TPGY medium were centrifuged to produce culture supernatants or were not centrifuged for use as whole culture samples. Half of the samples were treated with trypsin at 50 ug/ml for 30 minutes at 37° C. The activation process was stopped by adding soybean trypsin inhibitor at the final concentration of 100 μg/ml. 0.5 ml of each sample was injected intraperitoneally into each of two mice.

In mice injected with cultures of cells and spores in quantities which, had the organism been C. botulinum, would have contained about 400,000 lethal doses and no evidence of toxigenicity was observed.

### Example 5

### The Innate Immune Responses Elicited by Spores and Appendages

The present example shows that the injection of C. taeniosporum spores or isolated appendages only elicited very modest innate immune responses in mice.

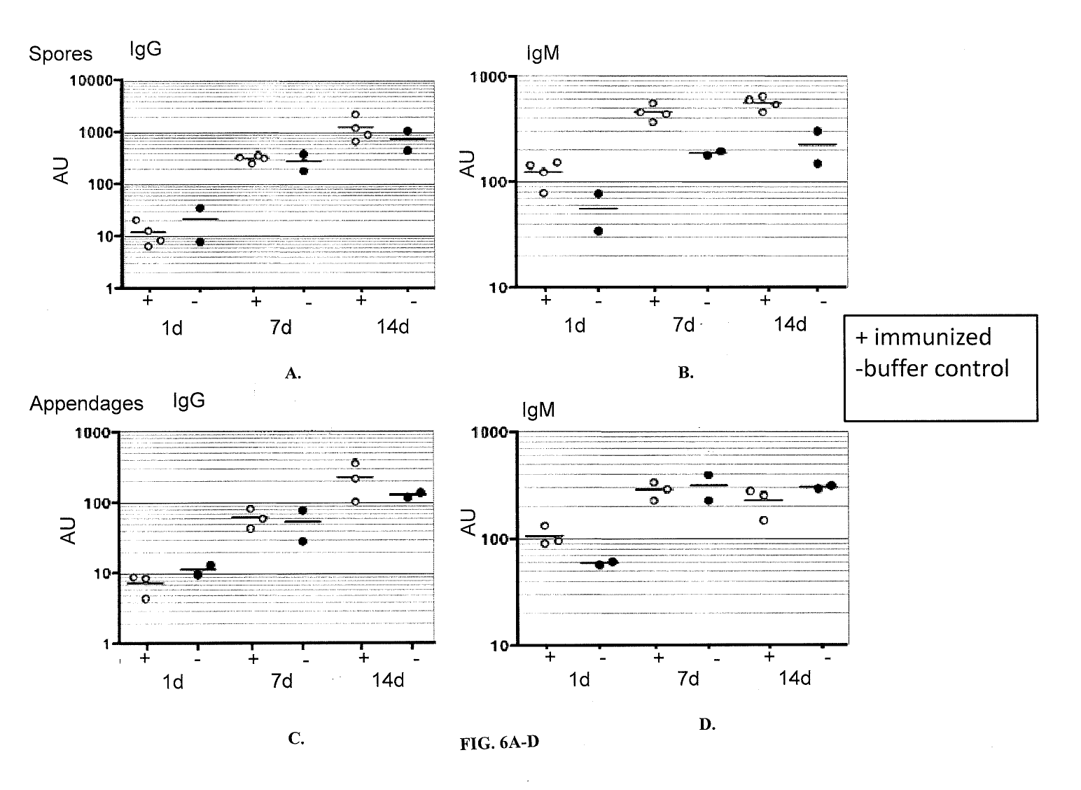

Each mouse was injected with either buffer, represented by open circles in FIGS. 6A-6D, or C. taeniosporum spores, represented by filled circles in FIGS. 6A and 6B, or isolated appendages, represented by filled circles in FIGS. 6C and 6D. FIGS. 6A and 6B show IgG or IgM responses of mice after injection with C. taeniosporum spores, respectively. FIGS. 6C and 6D show IgG or IgM responses of mice after injection with isolated appendages, respectively. IgG and IgM responses were measured by ELISA of sera drawn on the indicated days (d) and then plotted on the Y axis as Absorption Units (AU).

FIGS. 6A-6D illustrate that mice injected with either C. taeniosporum spores or isolated appendages exhibit similar IgG or IgM responses compared with mice injected with buffer only. These results demonstrate that mice are very well tolerant to the injections of spores or isolated appendages. Thus, the spores or appendages described in the present invention offer another technical advantage by providing a neutral platform to develop a vaccine composition comprising a spore displaying an antigenic peptide on its surface.

### Example 6

### Methods of Spore Surface Displays for Vaccine Development

The present example is a prophetic example presenting methods for producing an embodiment composition of a spore surface display system which may be used as a vaccine. For illustration purposes a vaccines for an enterohemorrhagic strain of Escherichia coli (EHEC) is described. However, this example does not limit the scope of this disclosure and other vaccines may be prepared for other organisms in light of the teachings herein.

The EHEC organism secretes Shiga-like toxins which efface the intestinal epithelium and are absorbed into circulation also causing the frequently fatal hemolytic uremic syndrome. In one embodiment, epitopes of a Shiga-like toxin 2 may be displayed on the appendages for use as an anti-EHEC vaccine.

Both genetic fusions and synthetic chemical cross-linking approaches may be used. Coding sequences for peptides corresponding to a Shiga-like toxin 2 epitope on exposed regions of Shiga toxin A and B subunits will be inserted into appendage protein genes at sites predicted to be highly solvent accessible. The appendage genes will be cloned under their natural promoter control on a multicopy plasmid or inserted into the chromosome after knockout of the wild-type appendage protein gene. This may prevent synthesis of the wild-type protein and to maximize incorporation of the fusion protein into appendages.

For chemical cross-linking, N-terminal maleimide-derivatized epitope peptides which will readily react with exposed nucleophilic cysteine thiol groups on the appendages may be used. Formation of correctly assembled, modified appendages will be confirmed by microscopy. Expression levels of the modified appendage proteins, their affinity for anti-Shiga-like toxin 2 peptide antibodies, and the immunogenicity of the modified appendages in mice will be assessed by serological techniques. The ability of antibodies produced in mice to neutralize Shiga-like toxin 2 in vitro and protection of mice against EHEC by immunization will be tested.

The present system may offer one or more of the following advantages. First, a spore appendage display system may be superior to bacterial or viral cell surface displays. The later require secretory signals and apparatus to transport the proteins synthesized in the cytoplasm to the cell exterior, whereas spore appendage proteins are not secreted by co-transported with any displayed protein. Appendage proteins are generally synthesized only late in sporulation within the mother cell cytoplasm and deposited onto the spore developing in the same cytoplasm. Bacterial surface displays are further limited by the fact that fusion proteins may restrict or prevent bacterial growth. In contrast, spore appendage proteins are synthesized only late in sporulation at the time when the mother cell is already destined for lysis. As fusion derivatives of appendage proteins are not synthesized during normal growth of the host and hence have no effect on growth or viability.

Second, C. taeniosporum is non-pathogenic, as shown by testing in mice. Third, spore display systems offer stability. For example, a vaccine of the disclosure may be either an intact spore with appendages co-displaying an antigenic epitope or may be an isolated appendage having an antigenic epitope. Both may be easily purified and stored in pure water where they are stable indefinitely (i.e., where spore germination is not possible) and no refrigeration would be required.

In some embodiments, living cells may be used if they colonize and survive in the gut for long periods. This may require also that the cells sporulate in the gut. This ability will be tested by oral administration of living C. taeniosporum cell to mice followed by collection and culture of feces to determine if viable cells survive in the intestine, collection and heating of feces before culture to test for viable spores of C. taeniosporum (which will survive the heat). Viable clostridia will be tested biochemically and genetically to determine if they are C. taeniosporum.

Fourth, in some embodiments a vaccine according to the disclosure may comprise combining an antigen with an adjuvant into one macrostructure, which may enhance its effectiveness.

In some embodiments, a vaccine of the disclosure may induce both IgA antibodies to inactivate Shiga-like toxins in the intestine at the mucosal level and IgG to inactive circulating toxin.

In some embodiments, a vaccine of the disclosure may be administered by systemically (by injection or other systemic methods as described in previous sections) or may be deliverable as an oral vaccine or delivered to the intestinal tract by gavage.

In some embodiments, a vaccine of the disclosure may be created as a super-polyvalent immunogen by incorporating different epitopes into the modified appendages. These may include different epitopes of the Shiga-like toxins or epitopes of a second protein known to be involved in pathogeniticy. For example, epitopes of the EHEC outer membrane protein, intimin, which is necessary for adhesion of this organism to the gut epithelium may be included.

### Example 7

### Methods of Using APEx in Combination with the Spore Surface Display System

The present example illustrates methods for using APEx to increase display efficiency of the spore surface display system. For illustration purposes, E. coli is described as a model system for appendages reconstruction and APEx selection. This example, however, does not limit the scope of this disclosure and other appropriate strains or systems may be employed in light of the teaching herein.

Appendage Reconstitution in E. coli: The fact that three small cytoplasm proteins (P29a/b, 269 residues and GP85, 387 residues) makes it feasible to establish appendage reconstitution in E. coli. By transferring P29a/b and GP85 into the E coli cytoplasm may facilitate the formation of pseudo-appendage in E. coli. An artificial operon of all three genes (P29a/b and GP85), each under IPTG control, will be constructed. The artificial operon will be insert into a suitable vector conventional in the art. An IPTG-inducible polycistronic vector in which appendage proteins will be co-expressed as either inner membrane associated or free in the periplasm in E. coli, will be constructed.

Identify Epitopes Presented on the Inner Membrane of E. coli based on binding affinity by APEx: A system that can identify epitopes presented on the inner membrane of E. coli on the basis of binding affinity by using the technique APEx has been developed (Harvey et al, Proc. Natl. Acad. ScL USA 101, 9193-9198 (2004); Jeong et al, Proc Natl. Acad Sci USA 104, 8247-8252 (2008), each incorporated by reference in its entirety).

By using APEx, one protein (e.g. G85) will be anchored on the periplasmic side of the inner membrane and its interacting partners (eg, p29a/b) will be expressed as soluble, epitope-inserted, periplasmic proteins. After removal of the outer membrane, periplasmic proteins, (including unbound epitope-integrated appendage complex members) are released into the extracellular fluid. If the epitope-integrated member(s) can bind to the membrane-anchored member, it remains associated with the spheroplast, forms the appendage and can be detected quantitatively by fluorescent anti-epitope mAb. Cells expressing epitope-modified appendages exhibiting different binding affinities can be readily distinguished by flow cytometry.

The APEx system can be used for isolation of affinity enhanced appendages from libraries of random insertion mutants. First, an epitope gene will be randomly inserted into an appendage protein gene by using a random insertion technique known in the art. For example, in vitro Tn5-mediated random epitope insertion can be used. The construction and analysis of short peptide/epitopes by insertional scanner linker mutagenesis (SLM) has been optimized in vitro by Epicentre Biotechnology using a modified Tn5 transposon that has an 1000-fold higher random insertional frequency than that of wt Tn5 (Goryshin and Reznikoff, J Biol Chem 273: 7367-7375 (1998), incorporated by reference in its entirety).

For the purposes of illustration, FLAG epitope will be used as an example to describe screening technique for high affinity epitope insertion. FLAG epitope gene will be randomly inserted into an appendage protein gene by using a modified Tn5 transposon. Performing triple simultaneous mutation of all 3 appendage genes on a single polycistronic plasmid would compromise the efficiency of the conventional reaction for randomization unless each C. taeniosporum appendage protein is amplified by a different drug selection. Thus, only one protein can be mutagenized at a time (Butterfield et al, Nucleic Acids Res. 30:2460-2469 (2002), incorporated by reference in its entirety). While the bulk of the non-epitopic portion of the artificial transposon will be deleted by cleavage and re-ligation at the unique Not I site, inserted FLAG peptides will retain 7 amino acid “mosaic” flanking sequences (Goryshin and Reznikoff, J Biol Chem 273: 7367-7375 (1998), incorporated by reference in its entirety). No strategy exists for mosaic elimination; this is a recognized limitation of the approach. (Butterfield, et al. (2002))

Following in vitro SLM, FLAG epitope insertional libraries of P29a/b and GP85 will be generated by individual cloning into the APEx polycistronic vector. At least 2×10⁶ independent transformants will be required. About 20-25 library clones will be sequenced to confirm that mutagenesis was random. Cells (˜2×10 will be sufficient) will be converted to spheroplasts, fluorescently labeled with the anti-FLAG-FITC conjugate, and sorted on a flow cytometer. The highest ˜10% of the sort will be isolated, and appendage genes will be rescued by PCR amplification. After APEx vector ligation and retransformation, a second round of sorting will be performed. The highest 1-10% of these cells will be plated at limiting dilution, and individual clones will be rescued by PCR and sequenced.

Anti-FLAG fluorescence of the spheroplasts will be proportional to the expression level, the number of appendage complexes per cell, and most importantly, to the affinity of the appendage complex to bind the antibody. To confirm affinity maturation, appendage proteins will be dissociated by centrifugation, and their kinetics of anti-FLAG monoclonal antibody binding will be determined by BIACore analysis.

The use of APEx technique in combination with the spore surface display system may offer one or more of the following advantages. First, absence this high throughput screening method of APEx, the choice of which appendage proteins to mutate and the optimal position for the mutation, such as the insertion mutation can only be predicted by structural analysis of appendage proteins. Following transformation into C. taeniosporum, high affinity FLAG insertions identified in E. Coli by APEx will be reconstituted into higher affinity appendages, compared with FLAG insertions designed based on the structural analysis of appendage proteins. Second, by using FLAG as epitope for APEx analysis, the best place to insert epitope into the appendage protein will be identified. The identified position can be used to insert other epitopes, such as Shiga-like toxin 2 epitope. By inserting an epitope into the identified optimal position, the fusion protein displayed on the surface of a spore will have a higher affinity to a target molecule, such as a ligand, an antibody.

Although several embodiments have been illustrated and described in detail, it will be recognized that substitutions and alterations are possible without departing from the spirit and scope of the present invention, as defined by the appended claims.

What is claimed is:

1. A composition comprising a bacterial spore derived from a Clostriduim sp. having at least one nucleic acid sequence encoding for at least one polypeptide operable to synthesize the polypeptide and express the at least one polypeptide on the surface of the spore.

2. The composition of claim 1, wherein the Clostriduim sp. is a Clostriduim taeniosporum.

3. The composition of claim 1, wherein the polypeptide is an antigen, an epitope, an enzyme, an antibody, an anticancer polypeptide, an apoptotic protein, a hormone, a therapeutic protein, a tissue marker, a cancer marker or any combination thereof.

4. The composition of claim 1, wherein the nucleic acid encoding the at least one polypeptide is engineered into an operon of the spore.

5. The composition of claim 4, wherein the operon encodes for one or more spore coat protein, one or more exosporia proteins, or one or more spore appendage protein.

6. The composition of claim 1, wherein the nucleic acid encoding the at least one polypeptide is under the control of a spore promoter.

7. The composition of claim 6, wherein the spore promoter also controls the expression of a spore coat protein, an exosporia protein or a spore appendage protein.

8. The composition of claim 7, wherein the spore appendage protein is selected form a GP85 glycoprotein, a P29a protein, a P29b protein, or combinations thereof.

9. The composition of claim 6, wherein the nucleic acid is under the control of a mother cell promoter.

10. The composition of claim 9, wherein the mother cell promoter is a late mother cell promoter.

11. The composition of claim 1, further comprising a pharmaceutical formulation.

12. The composition of claim 1, further defined as a spore-surface display system comprising a Clostriduim taeniosporum spore having at least one nucleic acid sequence encoding a polypeptide engineered under the control of a promoter that controls the expression of at least one carrier protein, the promoter operable to generate a fusion protein comprising the polypeptide and the carrier protein; wherein the fusion protein is assembled on the surface of the spore, thereby is displayed on the surface of the spore.

13.-19. (canceled)

20. The composition of claim 12, wherein the nucleic acid sequence is comprised in a plasmid.

21. The composition of claim 12, wherein the promoter is a spore promoter.

22. The composition of claim 12, wherein the carrier protein is a spore coat protein, an exosporial protein, or a spore appendage protein.

23. The composition of claim 12, wherein the polypeptide is an antigen, an epitope, an enzyme, an antibody, an anticancer polypeptide, an apoptotic protein, a hormone, a therapeutic protein, a tumor marker, a tissue marker, or a cancer marker.

24. The composition of claim 12, wherein the fusion protein comprises a Clostriduim taeniosporum spore appendage protein selected from the group consisting of a GP85 protein, a P29a protein, a P29b protein, an ortholog of a SpoVM protein, and combinations or fragments thereof.

25. The composition of claim 12, wherein said plasmid stably exists in the spore.

26. The composition of claim 12, wherein said plasmid incorporates into the genome of the spore.

27. The composition of claim 12, wherein said spore promoter is a mother cell promoter.

28. The composition of claim 27, wherein said spore promoter is a late mother cell promoter.

29. The compositions of claim 12, wherein said plasmid further comprises a general transcription promoter wherein said transcription promoter controls the expression of the fusion protein in E. coli.

30. The composition of claim 29, wherein said plasmid is engineered in E. coli prior to transformation into a Clostriduim taeniosporum spore.

31. The composition of claim 30, wherein said plasmid is engineered to insert a polypeptide gene into a carrier gene.

32. The composition of claim 31, wherein the carrier gene is a spore appendage gene.

33. A vaccine composition comprising a Clostriduim taeniosporum spore composition in accordance with claim 1, further defined as having at least one antigenic polypeptide expressed on a surface of the spore, the antigenic polypeptide encoded by a nucleic acid under the control of a promoter wherein the promoter also controls the expression of at least one carrier protein; and the carrier protein and the antigenic polypeptide are co-expressed and assembled as a fusion protein on the surface of the spore.

34.-46. (canceled)

47. A method for eliciting an immune response in a subject comprising administering to the subject a vaccine in accordance with claim 33
thereby eliciting an immune response to the antigenic peptide in the subject.

48. A method of screening for a biomolecule of interest comprising
a) providing a spore surface display composition expressing a polypeptide having the ability to interact with the biomolecule of interest and produce a detectable reaction following the interaction;
b) contacting the spore surface display composition with a sample suspected of having the biomolecule of interest under conditions that allow interaction of the polypeptide and the biomolecule of interest; and
c) detecting interaction of the polypeptide and the biomolecule of interest,
wherein detection of a detectable reaction indicates the presence of the biomolecule of interest in the sample suspected.

49. A method of catalyzing a reaction comprising:
a) expressing an enzyme on a spore surface display system that is operable to catalyze a biochemical or chemical reaction of interest wherein the enzyme is co-expressed with a spore carrier protein;
b) optionally immobilizing the spore surface display system onto a surface;
c) contacting the spore display system with a sample having a chemical or a biochemical agent of which catalytic conversion is desired, under conditions that allow interaction and catalysis of the chemical or the biochemical agent with the enzyme; and
d) detecting the catalytic conversion.

50. A method of screening for high affinity epitope insertions, comprising the steps of:
a) obtaining a gram negative bacterium that comprises an appendage gene that encodes and expresses an appendage protein, the expressed appendage protein being associated with the inner membrane or free in the periplasm of said bacterium;
b) randomly inserting an epitope gene of interest into the appendage gene to form a fused gene encoding fused polypeptides comprising epitope sequences fused with appendage gene sequences;
c) preparing therefrom a bacterial insertional library wherein members of said library express different fused polypeptides; and
d) screening the library to identify high affinity epitopes.

51.-55. (canceled)
